(19) United States
(12) Patent Application Publication
(10) Pub. No.: US 2018/0207005 A1
(43) Pub. Date: Jul. 26, 2018

(54) PROSTHETIC HAND

(71) Applicants: Chien-Wen CHEN, New Taipei City (TW); Chien-Cheng CHEN, New Taipei City (TW)

(72) Inventors: Chien-Wen CHEN, New Taipei City (TW); Chien-Cheng CHEN, New Taipei City (TW)

(21) Appl. No.: 15/877,013

(22) Filed: Jan. 22, 2018

(30) Foreign Application Priority Data
Jan. 23, 2017 (TW) 106102340
Nov. 22, 2017 (TW) 106140469

(51) Int. Cl. A61F 2/58; A61F 2/72; A61F 2/76

(52) U.S. Cl. CPC A61F 2/583; A61F 2002/6845; A61F 2/76; A61F 2/72

(57) ABSTRACT

A prosthetic hand includes a main frame, a hold unit and a drive unit. The hold unit includes a first finger, and a second finger. The first finger has a first proximal end portion, and a first distal end portion opposite to the first proximal end portion. The drive unit includes a drive mechanism and a first transmission mechanism. The first transmission mechanism is coupled to the drive mechanism and the first finger. The drive mechanism drives the first transmission mechanism to rotate the first finger about a first axis between a first closed position and a first open position, and to rotate the first finger about a second axis transverse to the first axis between the first open position and an eversion position.

### CROSS-REFERENCE TO RELATED APPLICATION

This application claims priority of Taiwanese Patent Application No. 106102340, filed on Jan. 23, 2017, and priority of Taiwanese Patent Application No. 106140469, filed on Nov. 22, 2017.

### FIELD

The disclosure relates to a prosthetic hand, and more particularly to a prosthetic hand that can be switched among various states.

### BACKGROUND

A conventional prosthetic hand includes two holding modules that are movable relative to each other to switch between a closed state and an open state for holding or releasing an object. However, the conventional prosthetic hand may not meet various needs of an amputee since it has only the hold and release functions.

### SUMMARY

Therefore, an object of the disclosure is to provide a prosthetic hand that can alleviate the drawback of the prior art.

According to the disclosure, the prosthetic hand includes a main frame, a hold unit and a drive unit. The main frame has an outer surface. The hold unit includes a first finger, and a second finger that corresponds in position to the first finger. The first finger has a first proximal end portion that is proximate to the outer surface, and a first distal end portion that is opposite to the first proximal end portion. The second finger is disposed on the main frame. The drive unit includes a drive mechanism and a first transmission mechanism. The drive mechanism is disposed on the main frame. The first transmission mechanism is disposed on the main frame, and is coupled to the drive mechanism and the first finger. The drive mechanism drives the first transmission mechanism to rotate the first finger about a first axis between a first closed position and a first open position, and to rotate the first finger about a second axis that is transverse to the first axis between the first open position and an eversion position. The first distal end portion of the first finger is proximate to the second finger when the first finger is at the first closed position. The first distal end portion of the first finger is distal from the second finger when the first finger is at the first open position. The first distal end portion of the first finger is distal from the outer surface when the first finger is at the eversion position.

### BRIEF DESCRIPTION OF THE DRAWINGS

Other features and advantages of the disclosure will become apparent in the following detailed description of the embodiment with reference to the accompanying drawings, of which:

FIG. 1 is a perspective view illustrating an embodiment of a prosthetic hand according to the disclosure;

FIG. 2 is another perspective view illustrating the embodiment;

FIG. 3 is side view illustrating the embodiment;

FIG. 4 is a fragmentary exploded perspective view illustrating a hold unit of the embodiment;

FIG. 5 is a fragmentary exploded perspective view illustrating a drive mechanism of the embodiment;

FIG. 6 is another fragmentary exploded perspective view illustrating the drive mechanism;

FIG. 7 is a fragmentary exploded perspective view illustrating a first transmission mechanism of the embodiment;

FIG. 8 is a fragmentary sectional view taken along line VIII-VIII in FIG. 25 and illustrating the first transmission mechanism;

FIG. 9 is another fragmentary sectional view taken along line IX-IX in FIG. 25 and illustrating the first transmission mechanism;

FIG. 10 is a perspective view illustrating a second transmission mechanism of the embodiment;

FIG. 11 is a fragmentary exploded perspective view illustrating the second transmission mechanism;

FIG. 12 is an exploded perspective view illustrating a transmission assembly of the embodiment;

FIG. 13 is a fragmentary sectional view illustrating the second transmission mechanism;

FIG. 14 is a fragmentary side view illustrating the transmission assembly;

FIG. 15 is a fragmentary exploded perspective view illustrating a third transmission mechanism of the embodiment;

FIG. 16 is a fragmentary schematic side view illustrating the hold unit in a closed state;

FIG. 17 is a fragmentary side view illustrating a second constraint member of the embodiment at a first pivoting position;

FIG. 18 is a fragmentary schematic side view illustrating the hold unit in the closed state;

FIG. 19 is a fragmentary schematic side view illustrating the drive mechanism and the third transmission mechanism of the embodiment;

FIG. 20 is a top view illustrating a main frame of the embodiment at a first twist position;

FIG. 21 is an enlarged view illustrating an intermittent gear set of the embodiment in a non-meshing state;

FIG. 22 is another fragmentary schematic side view illustrating the drive mechanism and the third transmission mechanism;

FIG. 23 is a fragmentary schematic side view illustrating the hold unit in a pointing state;

FIG. 24 is a fragmentary side view illustrating the second constraint member at a second pivoting position;

FIG. 25 is a fragmentary schematic side view illustrating the hold unit in an open state;

FIG. 26 is a fragmentary side view illustrating the second constraint member at a third pivoting position;

FIG. 27 is a fragmentary side view illustrating a third constraint member of the embodiment at a second rotating position;

FIG. 28 is an enlarged view illustrating the intermittent gear set being switched into a meshing state;

FIG. 29 is another fragmentary schematic side view illustrating the drive mechanism and the third transmission mechanism;

FIG. 30 is a fragmentary schematic side view illustrating the hold unit in an eversion state;

FIG. 31 is an enlarged view illustrating the intermittent gear set in the meshing state;

FIG. 32 is a fragmentary side view illustrating a first finger of the hold unit at an eversion position;

FIG. 33 is a fragmentary side view illustrating the second constraint member at a fourth pivoting position;

FIG. 34 is a fragmentary side view illustrating the third constraint member at a third rotating position;

FIG. 35 is a top view illustrating the main frame at a second twist position; and

FIG. 36 is a fragmentary schematic side view illustrating the hold unit being switched from the eversion state to the open state.

### DETAILED DESCRIPTION

Before the disclosure is described in greater detail, it should be noted that where considered appropriate, reference numerals or terminal portions of reference numerals have been repeated among the figures to indicate corresponding or analogous elements, which may optionally have similar characteristics.

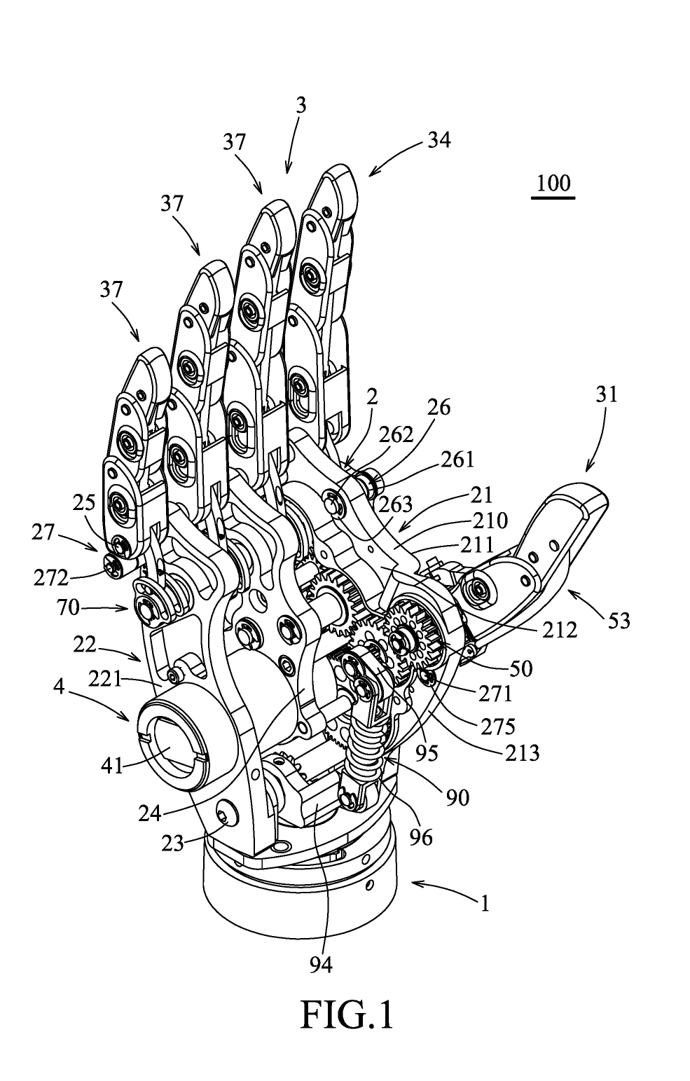

Referring to FIG. 1, the embodiment of the prosthetic hand 100 according to the disclosure includes a wrist frame 1, a main frame 2, a hold unit 3 and a drive unit 4. In one embodiment, the prosthetic hand 100 is exemplified by a right hand. For convenience sake, in the following paragraphs, the wrist portion the prosthetic hand 100 is denoted as the bottom portion, the finger portion of the prosthetic hand 100 is denoted as the top portion, the palm of the prosthetic hand 100 is denoted as the front portion, and the hand back of the prosthetic hand 100 is denoted as the rear portion.

Referring to FIGS. 1, 2 and 3, the main frame 2 includes a support plate 20, a first side plate 21, a second side plate 22, an axial post 23, a partition plate 24, an axial rod 25, a blocking assembly 26 and a limiting assembly 27.

The support plate 20 has a base plate portion 201 that is connected to the wrist frame 1, and two spaced-apart extending plate portions 202 that extend from a top surface of the base plate portion 201. The first side plate 21 covers one of the extending plate portions 202 and abuts against the base plate portion 201. The second side plate 22 abuts against an outer surface of the other one of the extending plate portions 202 that faces away from the one of the extending plate portions 202. The axial post 23 extends through the extending plate portions 202, the first side plate 21 and the second side plate 22 so that the first side plate 21 and the second side plate 22 are respectively connected to the extending plate portions 202 of the support plate 20.

The first side plate 21 has a first end surface 210 that is located at a top portion thereof, an outer surface 211 that is connected to the first end surface 210 and that faces away from the second side plate 22, an inner surface 212 that is connected to the first end surface 210 and that faces toward the second side plate 22, a second end surface 213 that is connected to the first end surface 210 and that faces forwardly, and a third end surface 214 that is connected to the first end surface 210 and that faces rearwardly. The second side plate 22 has an outer surface 221 that faces away from the first side plate 21, and an inner surface 222 that is opposite to the outer surface 221 and that faces toward the first side plate 21. The partition plate 24 is disposed between the first side plate 21 and the second side plate 22, and is spaced apart from the inner surface 212 of the first side plate 21 and the inner surface 222 of the second side plate 22. The axial rod 25 extends through the first side plate 21, the second side plate 22 and the partition plate 24, and has two opposite end portions respectively projecting out of the outer surface 211 of the first side plate 21 and the outer surface 221 of the second side plate 22. The axial rod 25 is disposed adjacent to the junction between the first end surface 210 and the third end surface 214.

The blocking assembly 26 includes a bearing 261, a positioning pin 262 and a clip 263. The bearing 261 is located at an outer side of the outer surface 211 of the first side plate 21, and is adjacent to the first end surface 210 of the first side plate 21. The positioning pin 262 extends through the bearing 261 and the first side plate 21, and abuts against an outer end of the bearing 261. The clip 263 is snapped onto the positioning pin 262 and abuts against the inner surface 212 of the first side plate 21, so as to prevent separation of the positioning pin 262 from the first side plate 21.

The limiting assembly 27 includes a first limiting member 271, an insertion pin 272, a second limiting member 273, a plurality of third limiting members 274, and a clip 275 (see FIG. 1). The first limiting member 271 is configured as a rod that extends through the first side plate 21. The first limiting member 271 is disposed adjacent to the second end surface 213 of the first side plate 21, and has opposite end portions respectively projecting out of the outer surface 211 and the inner surface 212 of the first side plate 21. The clip 275 is snapped onto the first limiting member 271 and abuts against the inner surface 212 of the first side plate 21, so as to prevent separation of the first limiting member 271 from the first side plate 21. The insertion pin 272 extends through the first side plate 21, the second side plate 22 and the partition plate 24, and has two opposite end portions respectively projecting out of the outer surface 211 of the first side plate 21 and the outer surface 221 of the second side plate 22. The insertion pin 272 is disposed adjacent to the junction between the first end surface 210 and the third end surface 214 of the first side plate 21, and is located below the axial rod 25. The second limiting member 273 is located at the outer side of the outer surface 211 of the first side plate 21, and is sleeved on the insertion pin 272. In this embodiment, there are three third limiting members 274. Each of the third limiting members 274 has a structure substantially the same as that of the second limiting member 273. One of the third limiting members 274 is disposed between the inner surface 212 of the first side plate 21 and the partition plate 24, and is sleeved on the insertion pin 272. Another one of the third limiting members 274 is disposed between the inner surface 222 of the second side plate 22 and the partition plate 24, and is sleeved on the insertion pin 272. The other one of the third limiting members 274 is located at the outer side of the outer surface 221 of the second side plate 22, and is sleeved on the insert ion pin 272. In one embodiment, the second limiting member 273 includes two jackets 276 that are sleeved on the insertion pin 272 and that are spaced apart from each other along the insertion pin 272. Each of the third limiting members 274 includes two jackets 277 that are sleeved on the insertion pin 272 and that are spaced apart from each other along the insertion pin 272.

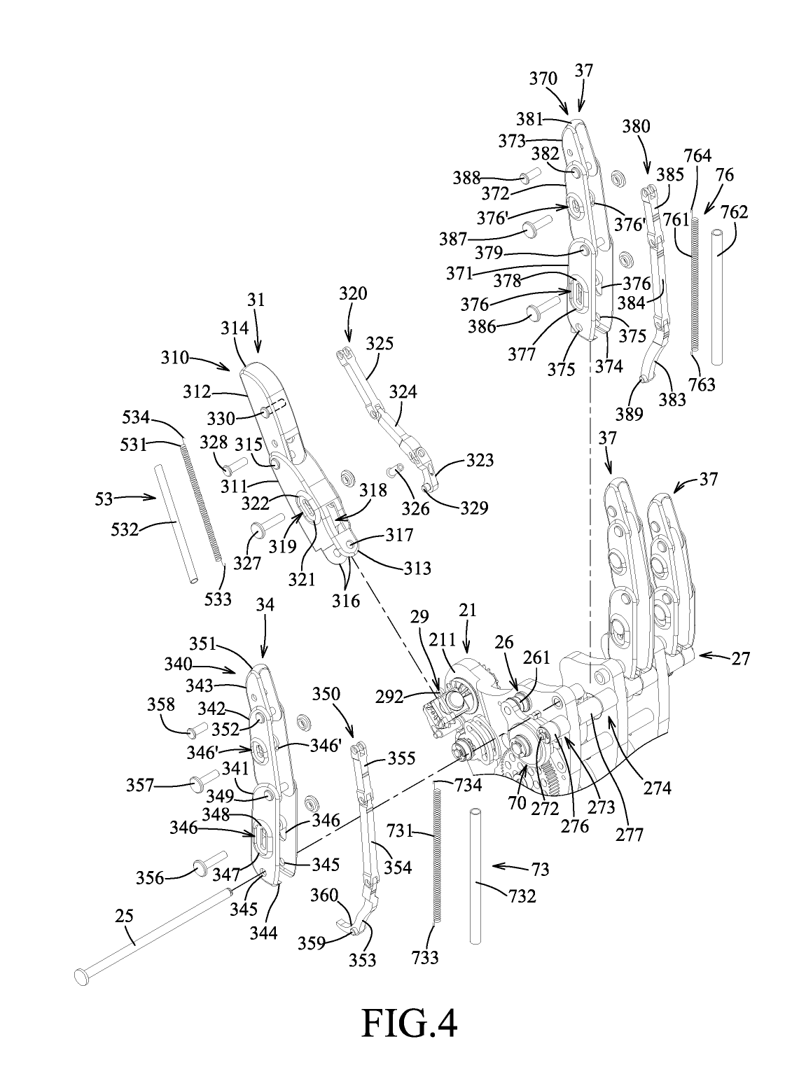

Referring to FIGS. 1, 3 and 4, the hold unit 3 includes a first finger 31, a second finger 34 and a plurality of third fingers 37. The first finger 31 serves as the thumb, is located at the outer side of the outer surface 211 of the first side plate 21, and is disposed adjacent to the junction between the first end surface 210 and the second end surface 213 of the first side plate 21. The first finger 31 includes a first finger body 310 and a first linkage 320. The first finger body 310 includes a first proximal phalange 311 and a first distal phalange 312. Each of the first proximal phalange 311 and the first distal phalange 312 is hollow and elongated. The first proximal phalange 311 has a first proximal end portion 313. The first distal phalange 312 has a first distal end portion 314. The first proximal end portion 313 is proximate to the outer surface 211 of the first side plate 21. The first distal end portion 314 is opposite to the first proximal end portion 313, and is distal from the outer surface 211 of the first side plate 21. A distal portion of the first proximal phalange 311 is pivotally connected to a proximal portion of the first distal phalange 312 by virtue of a pivoting axle 315. The first proximal end portion 313 has two end plates 316 that are spaced apart from each other substantially in a front-rear direction. Each of the end plates 316 is formed with a pivoting hole 317. A rear portion of the first proximal phalange 311 is formed with an opening 318 that permits the first linkage 320 to extend therethrough. The first proximal phalange 311 is formed with two first oblong grooves 319 (only one is visible in FIG. 4) respectively at two lateral sides thereof. Each of the first oblong grooves 319 extends in the extending direction of the first proximal phalange 311. Each of the first oblong grooves 319 has a proximal end 321 that is proximate to the first proximal end portion 313, and a distal end 322 that is distal from the first proximal end portion 313.

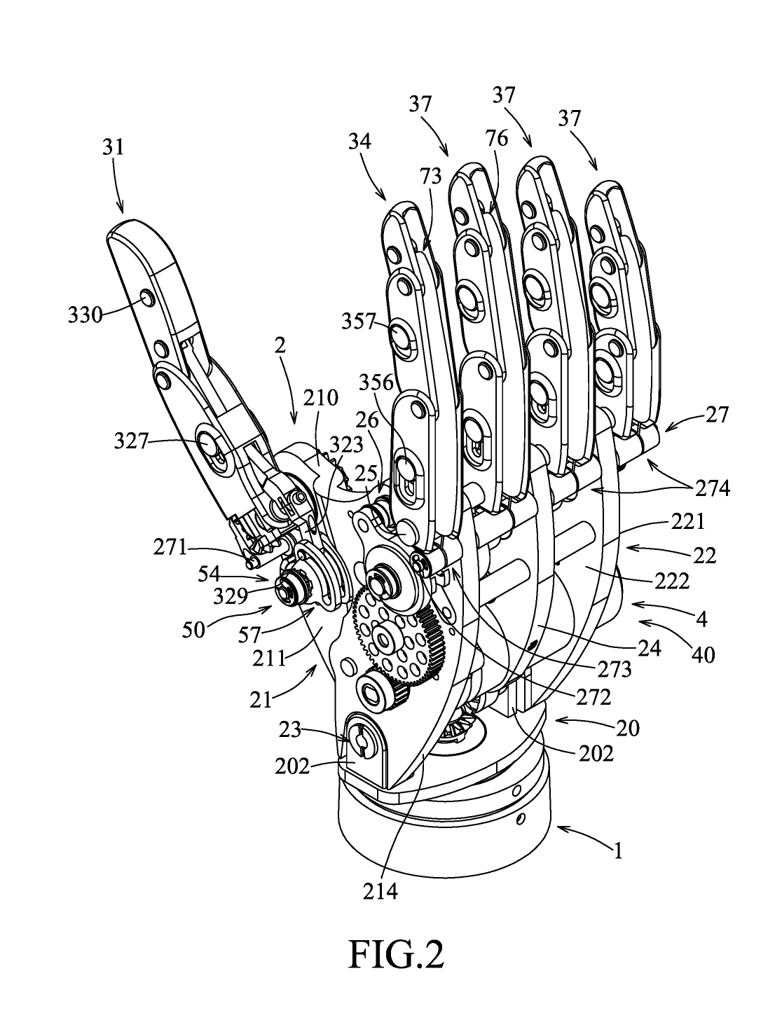

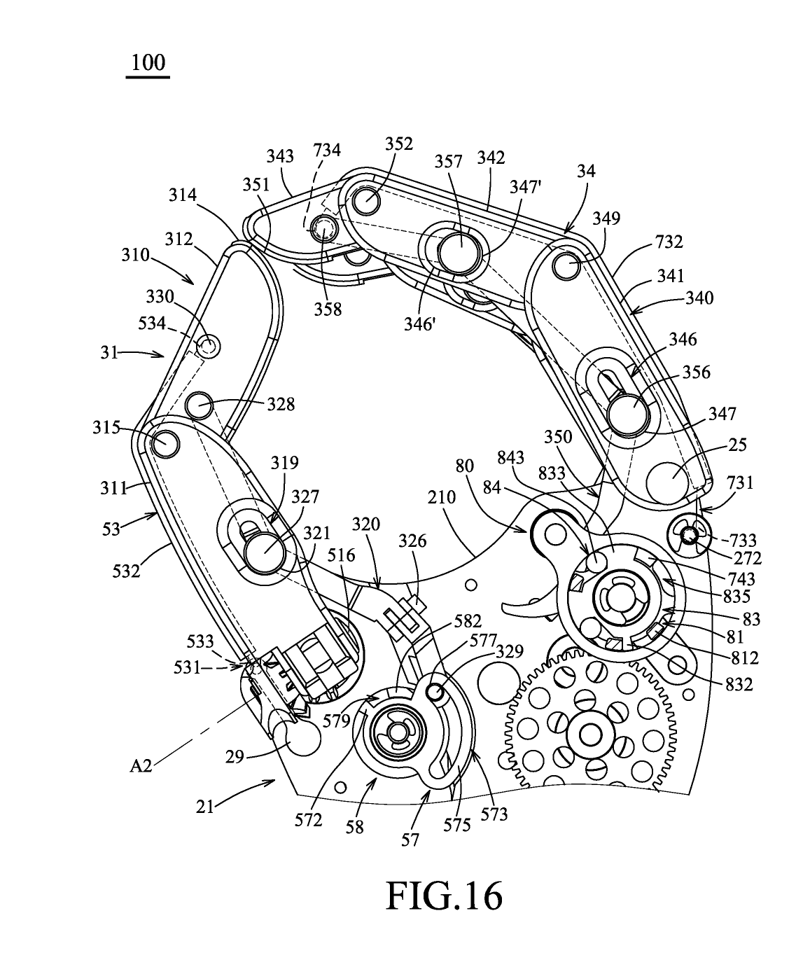

The first linkage 320 includes a first proximal link 323, a first intermediate link 324, a first distal link 325, a first proximal pivot axle 326, a first intermediate pivot axle 327, a first distal pivot axle 328 and a first pin 329. The first proximal link 323 is located at a rear side of the first proximal end portion 313 of the first proximal phalange 311. The first pin 329 extends through a proximal portion of the first proximal link 323, and has two opposite ends respectively projecting out of two opposite lateral sides of the first proximal link 323. The first intermediate link 324 extends through the opening 318 of the first proximal phalange 311, and has a proximal portion that is disposed out of the first proximal phalange 311 and that is located at the rear side of the first proximal end portion 313 of the first proximal phalange 311, and a distal portion that is disposed in the first proximal phalange 311. The proximal portion of the first intermediate link 324 is pivotally connected to a distal portion of the first proximal link 323 by virtue of the first proximal pivot axle 326, so the first intermediate link 324 is pivotable about the first proximal pivot axle 326 relative to the first proximal link 323. The first intermediate pivot axle 327 engages the first oblong grooves 319 of the first proximal phalange 311, and is slidable along the first oblong grooves 319. The first distal link 325 is disposed in the first proximal phalange 311 and the first distal phalange 312. A distal portion of the first intermediate link 324 is pivotally connected to a proximal portion of the first distal link 325 by virtue of the first intermediate pivot axle 327. A distal portion of the first distal link 325 is pivotally connected to the first distal phalange 312 by virtue of the first distal pivot axle 328. In this embodiment, the first intermediate pivot axle 327, the first distal pivot axle 328 and the first pin 329 are parallel to each other, and are orthogonal to the first proximal pivot axle 326. The first finger body 310 further includes a pin member 330 that is mounted to the first distal phalange 312 and that is disposed between the first distal end portion 314 and the first distal pivot axle 328. The pin member 330 is parallel to the first distal pivot axle 328. Since the first linkage 320 is connected to the first finger body 310 by virtue of the first intermediate pivot axle 327 and the first distal pivot axle 328, and since the first intermediate pivot axle 327 is movable along the first oblong grooves 319, the first linkage 320 can be driven by the drive unit 4 to actuate the first finger body 310, so as to move the first finger 31 between a first closed position (see FIG. 16) and a first open position (see FIG. 2).

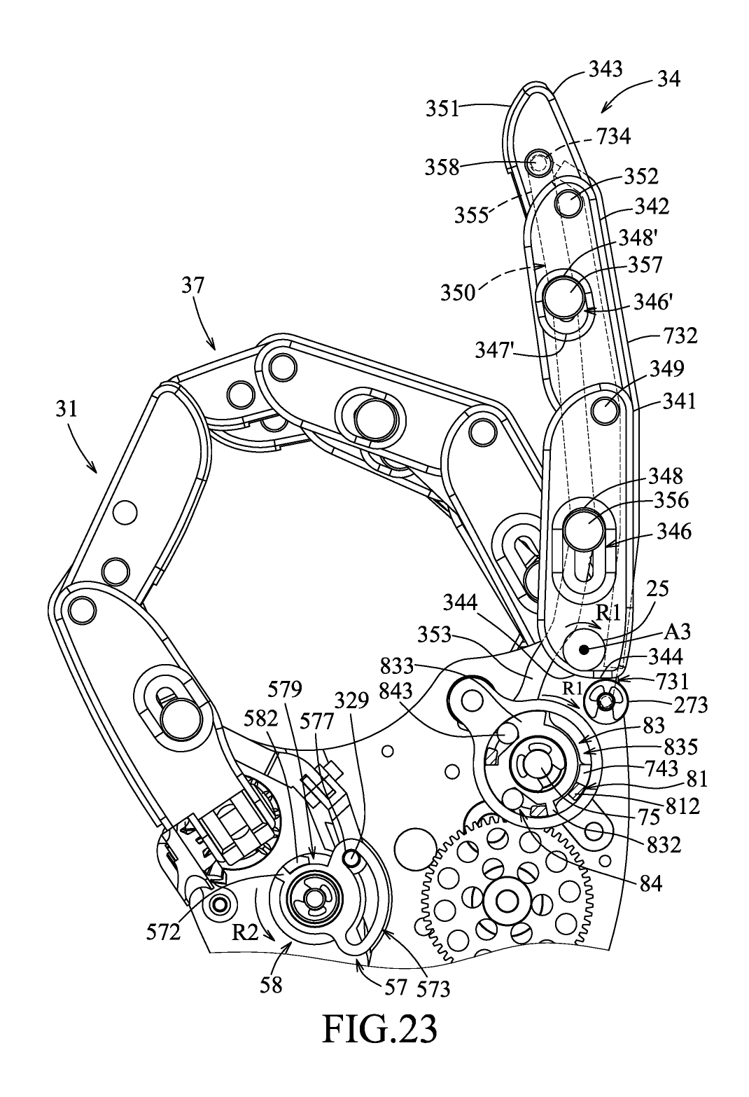

Referring to FIGS. 2, 3 and 4, the second finger 34 serves as the index finger, is rotatably mounted to the axial rod 25 of the main frame 2, and is located at the outer side of the outer surface 211 of the first side plate 21. The second finger 34 corresponds in position to the first finger 31, and is located at a rear side of the first finger 31. The second finger 34 includes a second finger body 340 and a second linkage 350. The second finger body 340 includes a second proximal phalange 341, a second intermediate phalange 342 and a second distal phalange 343. Each of the second proximal phalange 341, the second intermediate phalange 342 and the second distal phalange 343 is hollow and elongated. The second proximal phalange 341 has a second proximal end portion 344 that is located at the outer side of the outer surface 211 of the first side plate 21. The second limiting member 273 of the limiting assembly 27 is for blocking the second proximal end portion 344. The second proximal end portion 344 of the second proximal phalange 341 is formed with two pivoting holes 345 respectively at two opposite lateral sides thereof. The second proximal phalange 341 is further formed with two second oblong grooves 346 respectively at two opposite lateral sides thereof. The pivoting holes 345 permit the axial rod 25 to rotatably extend therethrough. Each of the second oblong grooves 346 is spaced apart from the pivoting holes 345, and extends in the extending direction of the second proximal phalange 341. Each of the second oblong grooves 346 has a proximal end 347 that is proximate to the second proximal end portion 344, and a distal end 348 that is distal from the second proximal end portion 344. A proximal portion of the second intermediate phalange 342 is pivotally connected to a distal portion of the second proximal phalange 341 by virtue of a pivoting axle 349. The second intermediate phalange 342 is formed with two second oblong grooves 346′ respectively at two opposite lateral sides thereof. Each of the second oblong grooves 346′ extends in the extending direction of the second intermediate phalange 342. With further reference to FIG. 23, each of the second oblong grooves 346′ has a proximal end 347′ that is proximate to the proximal portion of the second intermediate phalange 342, and a distal end 348′ that is distal from the proximal portion of the second intermediate phalange 342. The second distal phalange 343 has a second distal end portion 351. A proximal portion of the second distal phalange 343 is pivotally connected to a distal portion of the second intermediate phalange 342 by virtue of a pivoting axle 352.

The second linkage 350 includes a second proximal link 353, a second intermediate link 354, a second distal link 355, a second proximal pivot axle 356, a second intermediate pivot axle 357, a second distal pivot axle 358 and a second pin 359. The second proximal link 353 is partially disposed in the second proximal phalange 341, and has a proximal portion extending out of the second proximal end portion 344 of the second proximal phalange 341. The second proximal link 353 has a front surface 360 that abuts against the bearing 261 of the blocking module 26. In one embodiment, the front surface 360 may be arc-shaped. The second pin 359 extends through the proximal portion of the second proximal link 353, and has two opposite ends respectively projecting out of two opposite lateral sides of the second proximal link 353.

The second proximal pivot axle 356 engages the second oblong grooves 346 of the second proximal phalange 341, and is slidable along the second oblong grooves 346. The second intermediate pivot axle 357 engages the second oblong grooves 346′ of the second intermediate phalange 342, and is slidable along the second oblong grooves 346′. The second intermediate link 354 is disposed in the second proximal phalange 341 and the second intermediate phalange 342. A proximal portion of the second intermediate link 354 is pivotally connected to a distal portion of the second proximal link 353 by virtue of the second proximal pivot axle 356. The second distal link 355 is disposed in the second intermediate phalange 342 and the second distal phalange 343. A proximal portion of the second distal link 355 is pivotally connected to a distal portion of the second intermediate link 354 by virtue of the second intermediate pivot axle 357. A distal portion of the second distal link 355 is pivotally connected to the second distal phalange 343 by virtue of the second distal pivot axle 358. In this embodiment, the second proximal pivot axle 356, the second intermediate pivot axle 357, the second distal pivot axle 358 and the second pin 359 are parallel to each other. Since the second linkage 350 is connected to the second finger body 340 by virtue of the second proximal pivot axle 356, the second intermediate pivot axle 357 and the second distal pivot axle 358, since the second proximal pivot axle 356 is movable along the second oblong grooves 346 of the second proximal phalange 341, and since the second intermediate pivot axle 357 is movable along the second oblong grooves 346′ of the second intermediate phalange 342, the second linkage 350 can be driven by the drive unit 4 to actuate the second finger body 340, so as to move the second finger 34 between a second closed position (see FIG. 16) and a second open position (see FIG. 2).

In this embodiment, the number of the third fingers 37 is exemplified to be three. One of the third fingers 37 serves as the middle finger, is rotatably mounted to the axial rod 25 of the main frame 2, and is located between the inner surface 212 of the first side plate 21 and the partition plate 24 of the main frame 2. Another one of the third fingers 37 serves as the ring finger, is rotatably mounted to the axial rod 25 of the main frame 2, and is located between the inner surface 222 of the second side plate 22 and the partition plate 24 of the main frame 2. The other one of the third fingers 37 serves as the little finger, is rotatably mounted to the axial rod 25 of the main frame 2, and is located at the outer side of the outer surface 221 of the second side plate 22. The third limiting members 274 of the limiting assembly 27 respectively correspond to the third fingers 37. Since the third fingers 37 are structurally the same, and are only dimensionally different from each other, only one of the third fingers 37 is illustrated in detail in the following paragraphs.

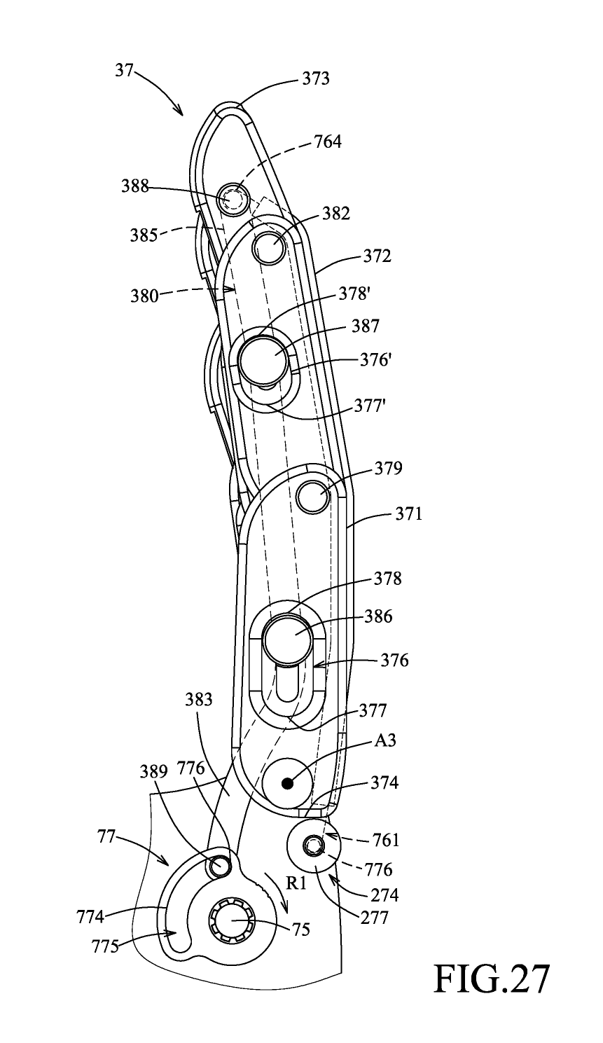

The third finger 37 includes a third finger body 370 and a third linkage 380. The third finger body 370 includes a third proximal phalange 371, a third intermediate phalange 372 and a third distal phalange 373. Each of the third proximal phalange 371, the third intermediate phalange 372 and the third distal phalange 373 is hollow and elongated. The third proximal phalange 371 has a third proximal end portion 374 that is proximate to the main frame 2 and that is for being blocked by the corresponding one of the third limiting members 274. The third proximal end portion 374 of the third proximal phalange 371 is formed with two pivoting holes 375 respectively at two opposite lateral sides thereof. The third proximal phalange 371 is further formed with two third oblong grooves 376 respectively at two opposite lateral sides thereof. The pivoting holes 375 permit the axial rod 25 to rotatably extend therethrough. Each of the third oblong grooves 376 is spaced apart from the pivoting holes 375, and extends in the extending direction of the third proximal phalange 371. Each of the third oblong grooves 376 has a proximal end 377 that is proximate to the third proximal end portion 374, and a distal end 378 that is distal from the third proximal end portion 374. A proximal portion of the third intermediate phalange 372 is pivotally connected to a distal portion of the third proximal phalange 371 by virtue of a pivoting axle 379. The third intermediate phalange 372 is formed with two third oblong grooves 376′ respectively at two opposite lateral sides thereof. Each of the third oblong grooves 376′ extends in the extending direction of the third intermediate phalange 372. With further reference to FIG. 27, each of the third oblong grooves 376′ has a proximal end 377′ that is proximate to the proximal portion of the third intermediate phalange 372, and a distal end 378′ that is distal from the proximal portion of the third intermediate phalange 372. The third distal phalange 373 has a third distal end portion 381. A proximal portion of the third distal phalange 373 is pivotally connected to a distal portion of the third intermediate phalange 372 by virtue of a pivoting axle 382.

The third linkage 380 includes a third proximal link 383, a third intermediate link 384, a third distal link 385, a third proximal pivot axle 386, a third intermediate pivot axle 387, a third distal pivot axle 388 and a third pin 389. The third proximal link 383 is partially disposed in the third proximal phalange 371, and has a proximal portion extending out of the third proximal end portion 374 of the third proximal phalange 371. The third pin 389 extends through the proximal portion of the third proximal link 383, and has two opposite ends respectively projecting out of two opposite lateral sides of the third proximal link 383.

The third proximal pivot axle 386 engages the third oblong grooves 376 of the third proximal phalange 371, and is slidable along the third oblong grooves 376. The third intermediate pivot axle 387 engages the third oblong grooves 376′ of the third intermediate phalange 372, and is slidable along the third oblong grooves 376′. The third intermediate link 384 is disposed in the third proximal phalange 371 and the third intermediate phalange 372. A proximal portion of the third intermediate link 384 is pivotally connected to a distal portion of the third proximal link 383 by virtue of the third proximal pivot axle 386. The third distal link 385 is disposed in the third intermediate phalange 372 and the third distal phalange 373. A proximal portion of the third distal link 385 is pivotally connected to a distal portion of the third intermediate link 384 by virtue of the third intermediate pivot axle 387. A distal portion of the third distal link 385 is pivotally connected to the third distal phalange 373 by virtue of the third distal pivot axle 388. In this embodiment, the third proximal pivot axle 386, the third intermediate pivot axle 387, the third distal pivot axle 388 and the third pin 389 are parallel to each other. Since the third linkage 380 is connected to the third finger body 370 by virtue of the third proximal pivot axle 386, the third intermediate pivot axle 387 and the third distal pivot axle 388, since the third proximal pivot axle 386 is movable along the third oblong grooves 376 of the third proximal phalange 371, and since the third intermediate pivot axle 387 is movable along the third oblong grooves 376′ of the third intermediate phalange 372, the third linkage 380 can be driven by the drive unit 4 to actuate the third finger body 370, so as to move the third finger 37 between a third closed position (see FIG. 16) and a third open position (see FIG. 2).

It should be noted that, although the number of the third fingers 37 is exemplified to be three, in a modification, there may be one, two, or more than three third fingers 37.

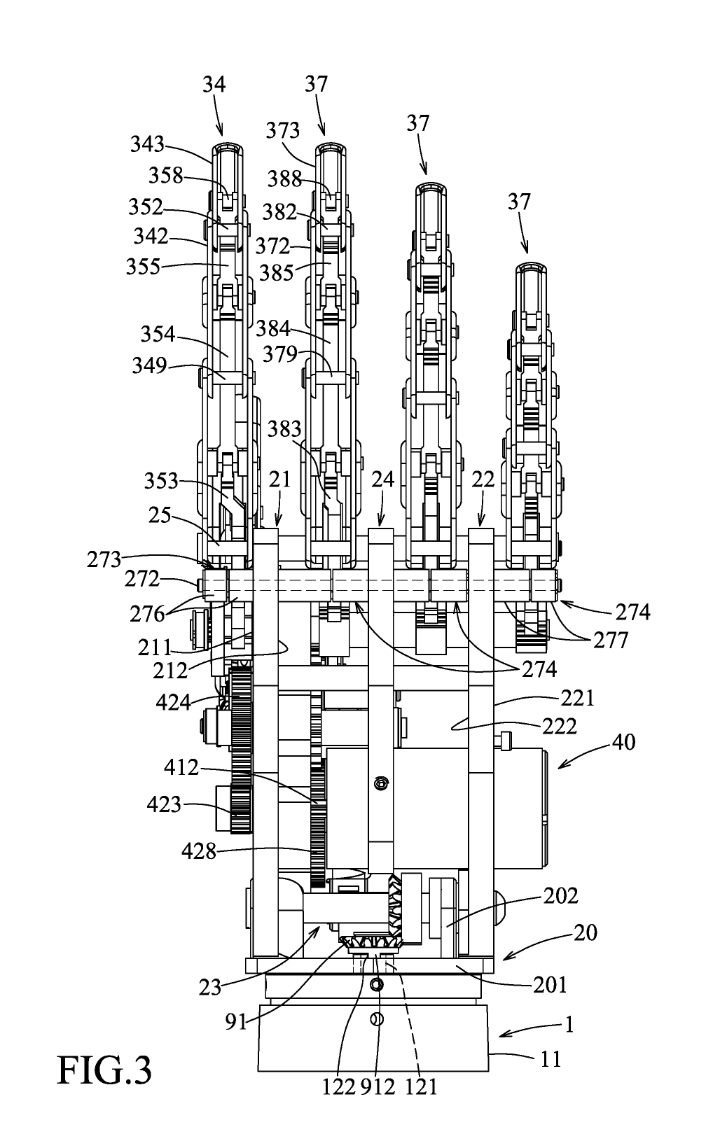

Referring to FIGS. 3, 5 and 6, the drive unit 4 is disposed on the main frame 2, and includes a drive mechanism 40, a first transmission mechanism 50, a second transmission mechanism 70 and a third transmission mechanism 90 (see FIG. 1). The drive mechanism 40 includes a drive motor 41, and a reduction gear train 42 that is coupled to and driven by the drive motor 41. The drive motor 41 is fixedly mounted to the second side plate 22 and the partition plate 24, and is located below the third fingers 37. In one embodiment, the drive motor 41 extends through the second side plate 22 and the partition plate 24. The drive motor 41 is electrically coupled to a first sensor (not shown) and a second sensor (not shown) by a plurality of wires (not shown). The first and second sensors are respectively disposed on inner and outer sides of the upper limb of a user. The first and second sensors receive electromyography signals of the muscle of the user for controlling the drive motor 41. The drive motor 41 includes an output shaft 411 that extends toward the inner surface 212 of the first side plate 21. The output shaft 411 is configured as a toothed shaft, and has a first toothed shaft section 412, and a second toothed shaft section 413 that extends from a distal end of the first toothed shaft section 412.

The reduction gear train 42 includes a first gear 421, a second gear 422, a third gear 423, a fourth gear 424, a fifth gear 425, a sixth gear 426 and a seventh gear 427. The first gear 421 is rotatably mounted to a first mount rod 281 of the main frame 2 that is mounted to the first side plate 21 and the partition plate 24. The first gear 421 has a large gear section 428 and a small gear section 429 that are coaxially arranged. The large gear section 428 of the first gear 421 meshes with the first toothed shaft section 412 of the output shaft 411 of the drive motor 41. The number of teeth of the large gear section 428 of the first gear 421 is greater than that of the first toothed shaft section 412 of the output shaft 411. The small gear section 429 of the first gear 421 is located at one side of the large gear section 428 proximate to the inner surface 212 of the first side plate 21. Rotation of the output shaft 411 of the drive motor 41 drives the first gear 421 to rotate at a speed lower than that of the output shaft 411.

The second gear 422 has a gear section 430 and an axle section 431 that are coaxially arranged. The gear section 430 of the second gear 422 meshes with the small gear section 429 of the first gear 421. A side surface of the gear section 430 of the second gear 422 is formed with a retaining space 432 for retaining a bearing 433 that is sleeved on the second toothed shaft section 413 of the output shaft 411 of the drive motor 41. Since the bearing 433 is disposed between the gear section 430 of the second gear 422 and the second toothed shaft section 413 of the output shaft 411, the second gear 422 and the output shaft 411 of the drive motor 41 are rotatable relative to each other. The number of teeth of the gear section 430 of the second gear 422 is greater than that of the small gear section 429 of the first gear 421, so rotation of the first gear 421 drives the second gear 422 to rotate at a speed lower than that of the first gear 421. The axle section 431 of the second gear 422 extends from an opposite side surface of the gear section 430 distal from the retaining groove 432, and rotatably extends through the first side plate 21 to project out of the outer surface 211 of the first side plate 21. The third gear 423 is disposed at the outer side of the first side plate 21, and is co-rotatablv mounted to the axle section 431 of the second gear 422, so as to be co-rotatable with the second gear 422.

The reduction gear train 42 further includes a connecting shaft 434 that is rotatably mounted to the first side plate 21 and the partition plate 24 and that projects out of the outer surface 211 of the first side plate 21. The fourth gear 424 is disposed at the outer side of the first side plate 21, is co-rotatably mounted to the connecting shaft 434, and meshes with the third gear 423. The fifth gear 425 is disposed between the inner surface 212 of the first side plate 21 and the partition plate 24, and is co-rotatably mounted to the connecting shaft 434. The number of teeth of the fourth gear 424 is greater than that of the third gear 423, so rotation of the third gear 423 drives the fourth gear 424 and the fifth gear 425 to rotate at a speed lower than that of the third gear 423.

The sixth gear 426 is rotatably mounted to a second mount rod 282 of the main frame 2 that is mounted to the first side plate 21 and the partition plate 24, and meshes with the fifth gear 425. The seventh gear 427 is rotatably mounted to a third mount rod 283 of the main frame 2 that is mounted to the first side plate 21 and the partition plate 24, and meshes with the sixth gear 426.

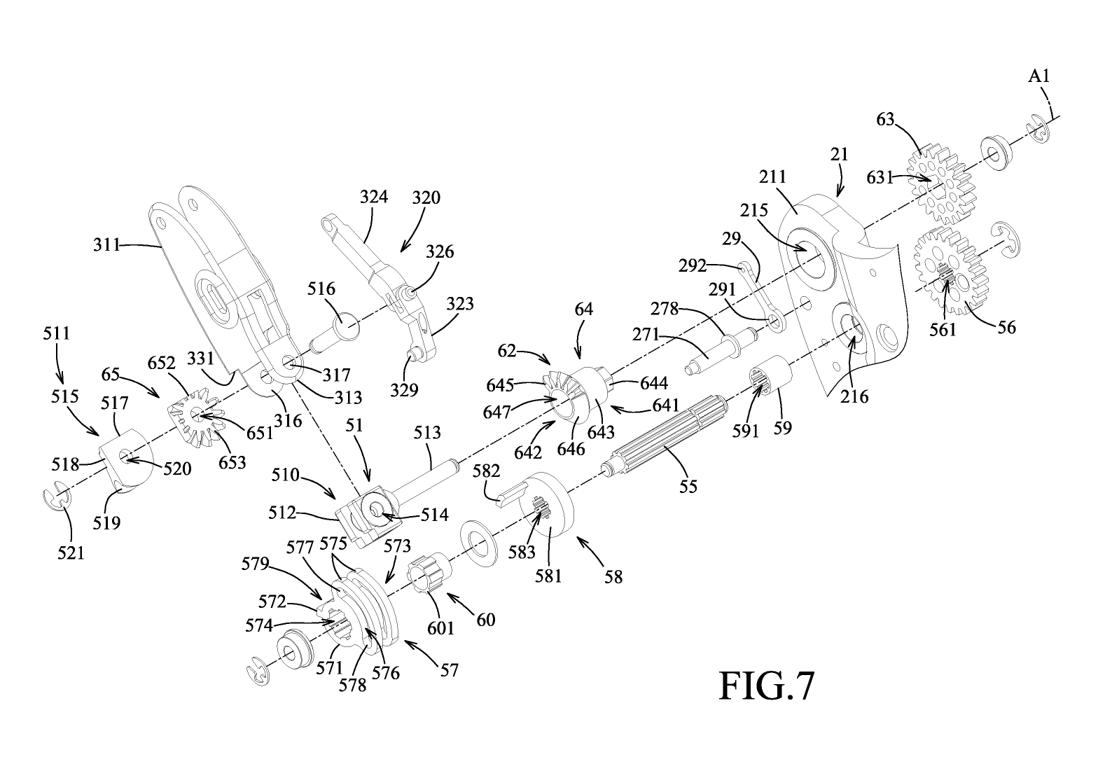

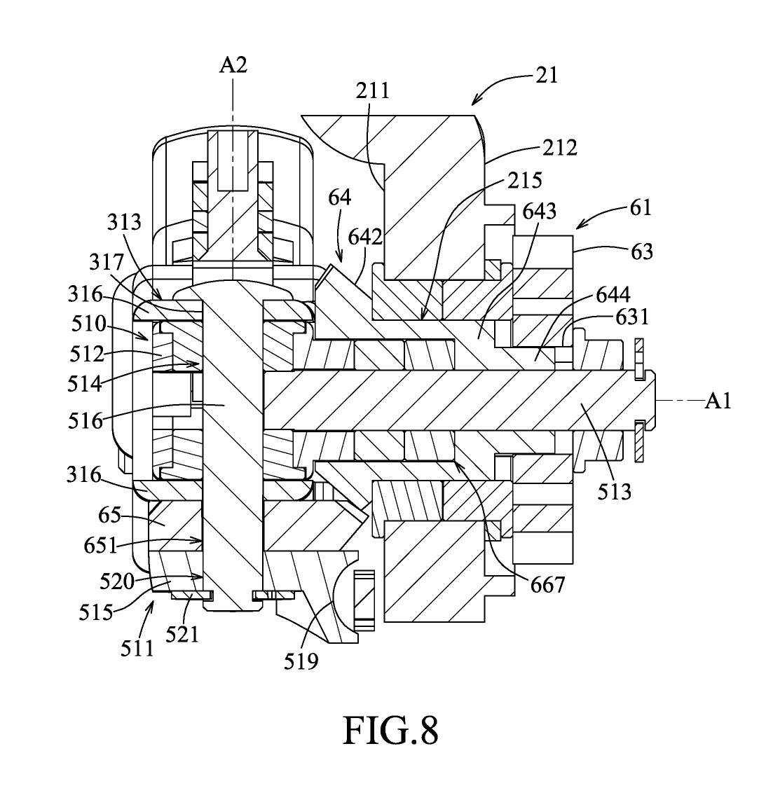

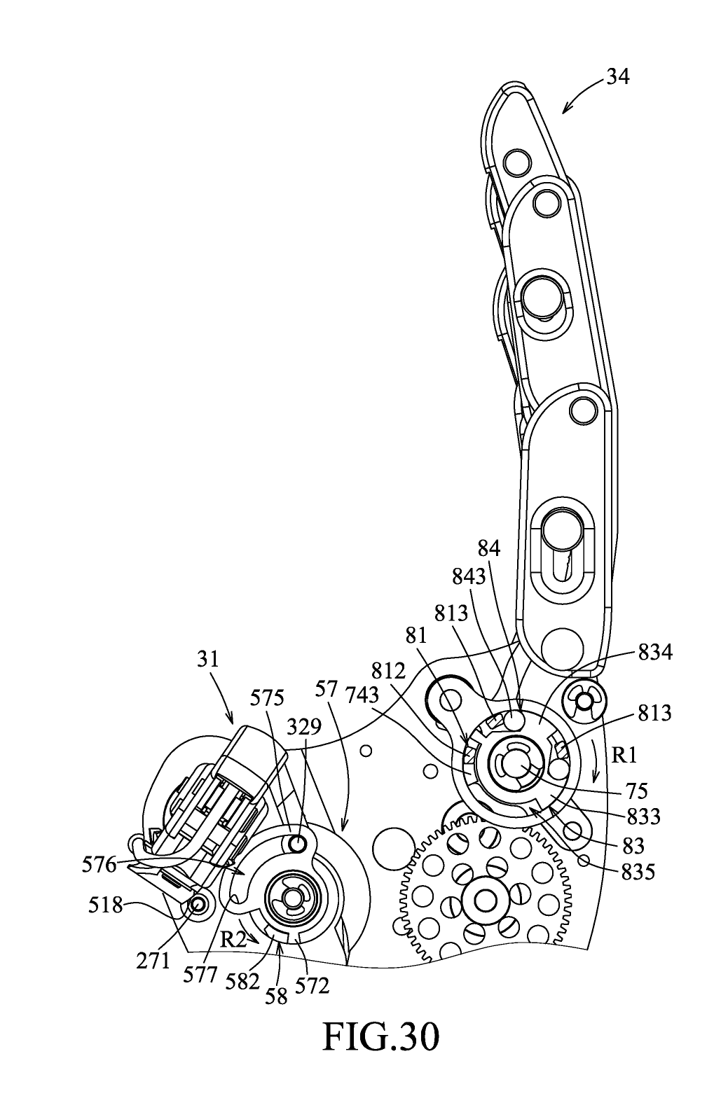

Referring FIGS. 2, 7 and 8, the first transmission mechanism 50 is disposed on the main frame 2, and is coupled to the drive mechanism 40 and the first finger 31. The drive mechanism 40 is operable to drive the first transmission mechanism 50, so as to rotate the first finger 31 about a first axis (A1, see FIG. 7) between the first closed position (see FIG. 16) and the first open position (see FIG. 2), and to rotate the first finger 31 about a second axis (A2, see FIG. 8) that is perpendicular to the first axis (A1) between the first open position and an eversion position (see FIG. 30). When the first finger 31 is at the first closed position, the first distal end portion 314 of the first finger 31 is proximate to the second finger 34. When the first finger 31 is at the first open position, the first distal end portion 314 of the first finger 31 is distal from the second finger 34. When the first finger 31 is at the eversion position, the first finger 31 extends in a direction away from the outer surface 211 of the first side plate 21, and the first distal end portion 314 of the first finger 31 is distal from the outer surface 211 of the first side plate 21.

The first transmission mechanism 50 includes a hinge 51, a first bias assembly 53 (see FIGS. 1 and 4) and a constraint assembly 54 (see FIG. 2). The hinge 51 is rotatably mounted to the first side plate 21 of the main frame 2, and is connected to the first proximal end portion 313 of the first finger 31. The hinge 51 includes a first pivoting module 510 and a second pivoting module 511. The first pivoting module 510 includes an axle block 512 that is located at the outer side of the first side plate 21, and a first pivot axle 513 that is mounted to the axle block 512 and that extends toward the first side plate 21. The axle block 512 is disposed between the end plates 316 of the first proximal end portion 313 of the first finger 31, and is formed with an axle hole 514 that is aligned with the pivoting holes 317 of the end plates 316. The first pivot axle 513 extends through an axle hole 215 of the first side plate 21, and defines the first axis (A1). The second pivoting module 511 includes a plate body 515 and a second pivot axle 516. The plate body 515 has a first side surface 517, a second side surface 518 that is connected to the first side surface 517 and that is perpendicular to the first side surface 517, and an arc-shaped surface 519 that interconnects the first side surface 517 and the second side surface 518. The plate body 515 is formed with a pivoting hole 520. The first proximal end portion 313 of the first finger 31 has a proximal surface 331 (see FIG. 7) that is perpendicular to a lower one of the end plates 316. The first side surface 517 of the plate body 515 abuts against the proximal surface 331 of the first proximal end portion 313 of the first finger 31. The arc-shaped surface 519 is substantially semicircular, and defines an arc-shaped groove. The first limiting member 271 is for blocking the arc-shaped surface 519 or the second side surface 518 of the plate body 515. The second pivot axle 516 extends through the pivoting holes 317 of the end plates 316, the axle hole 514 of the axle block 512 and the pivoting hole 520 of the plate body 515. The second pivot axle 516 projects out of a bottom end of the plate body 515. A clip 521 is snapped onto the second pivot axle 516 and abuts against the bottom end of the plate body 515, so as to prevent separation of the second pivot axle 516 from the end plates 316 of the first finger 31, the axle block 512 and the plate body 515. The second pivot axle 516 defines the second axis (A2).

Referring to FIGS. 1, 4 and 7, the first bias assembly 53 is coupled to the main frame 2 and the first finger 31 for resiliently biasing the first finger 31 to move toward the first open position. The main frame 2 further includes an elongated support arm 29. The support arm 29 is formed with a first hole 291, and a second hole 292 that is spaced apart from the first hole 291. The first limiting member 271 extends through the first hole 291 of the support arm 29, and has a flange portion 278 that cooperates with the outer surface 211 of the first side plate 21 to hold the support arm 29 therebetween. The first bias assembly 53 includes a first spring 531 and a first sheath 532. The first spring 531 is configured as an extension spring. The first sheath 532 is made of an elastic material such as rubber or silicone. The first sheath 532 is sleeved on the first spring 531, and abuts against a front end of the first finger 31. The first spring 531 has a first connecting end 533, and a second connecting end 534 that is opposite to the first connecting end 533. The first connecting end 533 and the second connecting end 534 are respectively exposed from two opposite ends of the first sheath 532. The first connecting end 533 of the first spring 531 is connected to the support arm 29 via the second hole 292. The second connecting end 534 of the first spring 531 is connected to the pin member 330 of the first finger 31. The first spring 531 resiliently biases the first finger 31 to move toward the first open position.

Referring to FIGS. 2, 4, 7 and 8, the constraint assembly 54 is coupled to the drive mechanism 40, and is for constraining the first linkage 320. The drive mechanism 40 is operable to drive the constraint assembly 54 to move the first linkage 320, so that the first finger 31 can be biased by the first bias assembly 53 to move from the first closed position to the first open position, or be driven by the constraint assembly 54 to move from the first open position to the first closed position via the first linkage 320.

Referring to FIGS. 2, 5, 7 and 9, the constraint assembly 54 includes a first transmission shaft 55, a first transmission gear 56, a first constraint member 57, a poking member 58, a first bushing 59 and a second bushing 60. The first transmission shaft 55 is parallel to the first axis (A1), and has an outer spline. The first bushing 59 is rotatably mounted in a shaft hole 216 of the first side plate 21, and has an inner spline 591. An outer surrounding surface of the first bushing 59 is smooth. The first bushing 59 is sleeved on the first transmission shaft 55, and is co-rotatable with the first transmission shaft 55 by virtue of engagement between the inner spline 591 thereof and the outer spline of the first transmission shaft 55. By such, the first transmission shaft 55 is smoothly rotatable relative to the first side plate 21. The first transmission gear 56 is located at the inner side of the inner surface 212 of the first side plate 21, and has an inner spline 561. The first transmission gear 56 is sleeved on the first transmission shaft 55, and is co-rotatable with the first transmission shaft 55 by virtue of engagement between the inner spline 561 thereof and the outer spline of the first transmission shaft 55. The first transmission gear 56 meshes with the sixth gear 426 of the drive mechanism 40, so that rotation of the sixth gear 426 drives rotation of the first transmission gear 56 and the first transmission shaft 55.

The second bushing 60 is rotatably sleeved on the first transmission shaft 55, and is located at the outer side of the outer surface 211 of the first side plate 21. The second bushing 60 has a smooth inner surrounding surface, and an outer spline 601. The first constraint member 57 has a first barrel 571, a first protruding block 572 and a first constraint block 573. The first barrel 571 is sleeved on the second bushing 60, and has an inner spline 574 that engages the outer spline 601 of the second bushing 60, so that the first barrel 571 is smoothly rotatable relative to the first transmission shaft 55. The first protruding block 572 and the first constraint block 573 are disposed on an outer surrounding surface of the first barrel 571, and are angularly spaced apart from each other. The first constraint block 573 includes two first limiting plates 575 that are spaced apart from each other in the axial direction of the first transmission shaft 55. The first limiting plates 575 are respectively located at the opposite lateral sides of the first proximal link 323 of the first linkage 320 for limiting wobble movement of the first proximal link 323. Each of the first limiting plates 575 is formed with an arc-shaped guide groove 576. The first pin 329 of the first linkage 320 has two opposite ends respectively engaging the guide grooves 576 of the first limiting plates 575, and is movable along the guide grooves 576. Each of the first limiting plates 575 has a first stop surface 577 that is located at an end of the guide groove 576 thereof proximate to the first protruding block 572, and an end surface 578 that is located at the other end of the guide groove 576 distal from the first protruding block 572. The first stop surfaces 577 of the first limiting plates 575 serve to block or push the first pin 329. The first constraint member 57 further has an arc-shaped groove 579 that is defined between the first protruding block 572 and the first constraint block 573.

The poking member 58 has a wheel body 581 and a poking arm 582. The wheel body 581 is sleeved on the first transmission shaft 55, and is located between the first side plate 21 and the first constraint member 57. The wheel body 581 has an inner spline 583 that engages the outer spline of the first transmission shaft 55, so that the poking member 58 is co-rotatable with the first transmission shaft 55. The poking arm 582 is disposed on an outer periphery of the wheel body 581, and extends into the arc-shaped groove 579 of the first constraint member 57. The poking arm 582 serves to push the first protruding block 572 or the first constraint block 573 to rotate the first constraint member 57 for driving the first pin 329 of the first linkage 320. In this embodiment, the poking arm 582 has an arc-shaped cross-section that has an arc length smaller than the arc length of the arc-shaped groove 579 of the first constraint member 57

Referring to FIGS. 7 and 8, the first transmission mechanism 50 further includes a turning assembly 61. The turning assembly 61 includes an intermittent gear set 62 and a connecting gear 63. The intermittent gear set 62 is disposed between the first side plate 21 and the hinge 51, and is connected to the first proximal end portion 313 of the first finger 31 for intermittently turning the first finger body 310 of the first finger 31 about the second axis (A2). The connecting gear 63 meshes with the first transmission gear 56 for driving the intermittent gear set 62. The intermittent gear set 62 includes an active gear 64 and a passive gear 65. The active gear 64 is rotatably mounted to the axle hole 215 of the first side plate 21, is disposed between the first side plate 21 and the hinge 51, and is co-rotatable with the connecting gear 63. The active gear 64 has a hollow axle 641 and a gear body 642 that are coaxially arranged. The hollow axle 641 has a first axle section 643 that has a circular cross-section, and a second axle section 644 that extends from an axial end of the first axle section 643 and that has a noncircular cross-section. The gear body 642 is connected to the other axial end of the first axle section 643, and is located between the first side plate 21 and the axle block 512 of the hinge 51. The gear body 642 of the active gear 64 is configured as a sector gear, and has a toothed sector 645 and a non-toothed sector 646. The hollow axle 641 and the gear body 642 cooperatively define a pivoting hole 647 that permits the first pivot axle 513 of the first pivoting module 510 of the hinge 51 to rotatably extend therethrough, so that the active gear 64 and the first pivoting module 510 of the hinge 51 are rotatable relative to each other.

The passive gear 65 is sleeved on the second pivot axle 516 of the hinge 51, and is sandwiched between the lower one of the end plates 316 of the first finger 31 and the plate body 515 of the hinge 51. The passive gear 65 is formed with a pivoting hole 651 that permits the second pivot axle 516 of the hinge 51 to extend therethrough. The passive gear 65 has an annular gear section 653, and a truncated surface 652 that is formed at a radial end of the annular gear section 653. The truncated surface 652 abuts against the proximal surface 331 of the first proximal end portion 313 of the first finger 31, such that the passive gear 65 is co-rotatable with the first proximal end portion 313 of the first finger 31. The annular gear section 653 of the passive gear 65 is able to mesh with the toothed sector 645 of the active gear 64.

The connecting gear 63 is sleeved on the hollow axle 641 of the active gear 64, and is formed with a non-circular hole 631 that is engaged fittingly with the second axle section 644 of the hollow axle 641, so that the active gear 64 is co-rotatable with the connecting gear 63. The connecting gear 63 is located at the inner side of the inner surface 212 of the first side plate 21, and meshes with the first transmission gear 56, so that rotation of the first transmission gear 56 drives rotation of the connecting gear 63 and the active gear 64.

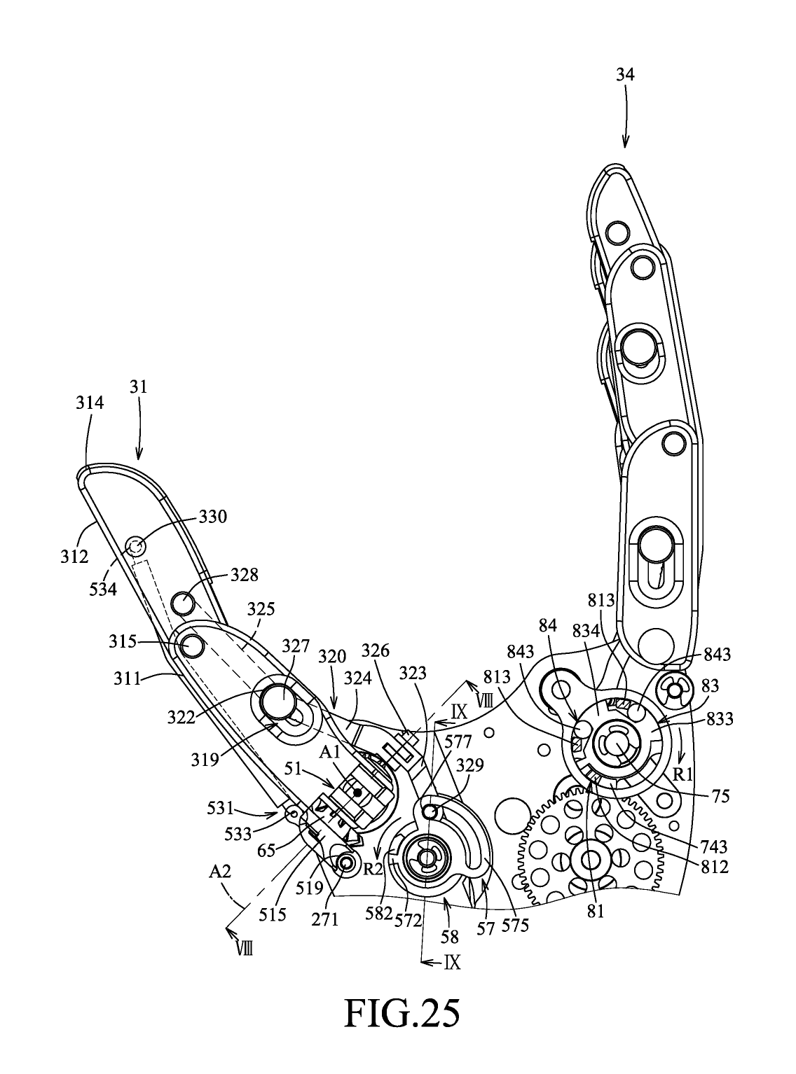

The intermittent gear set 62 is operable to switch between a non-meshing state in which the toothed sector 645 of the active gear 64 is separated from the annular gear section 653 of the passive gear 65, and a meshing state in which the toothed sector 645 of the active gear 64 meshes with the annular gear section 653 of the passive gear 65. The connecting gear 63 drives rotation of the active gear 64 to switch the intermittent gear set 62 between the non-meshing state and the meshing state, so that the active-gear 64 intermittently drives rotation of the passive gear 65. When the intermittent gear set 62 is in the non-meshing state, the passive gear 65 is not driven by the active gear 64. When the intermittent gear set 62 is in the meshing state, the passive gear 65 can be driven by the active gear 64 to rotate about the second axis (A2), so as to move the first finger 31 between the first open position (see FIGS. 2 and 25) and the eversion position (see FIG. 30).

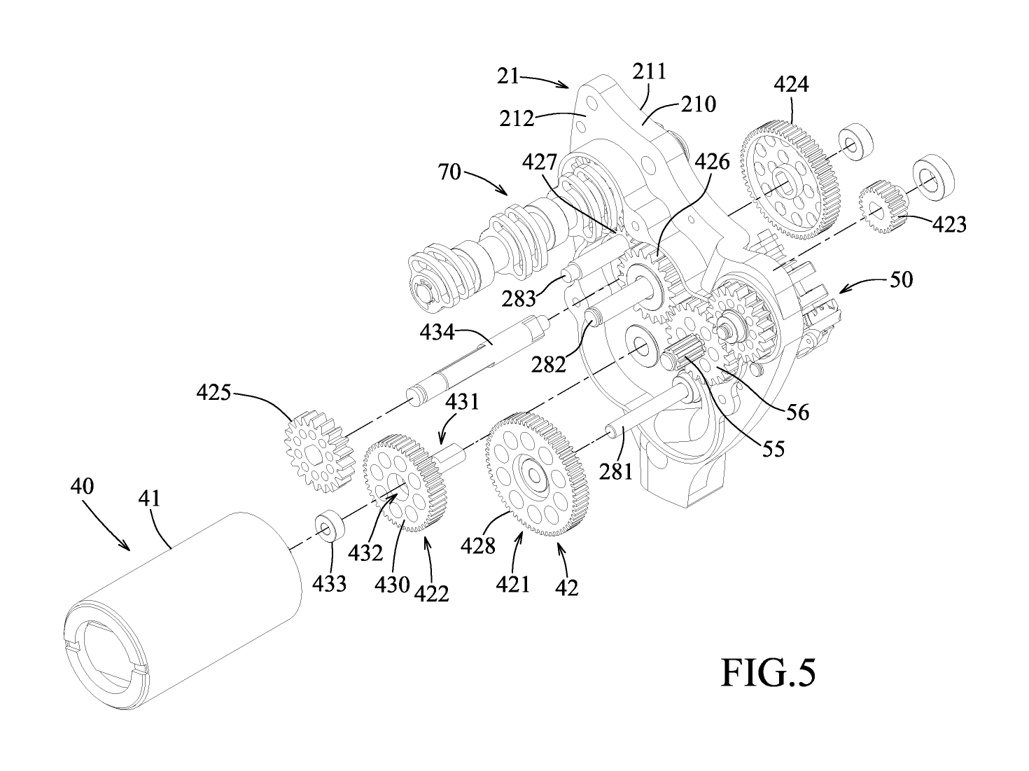

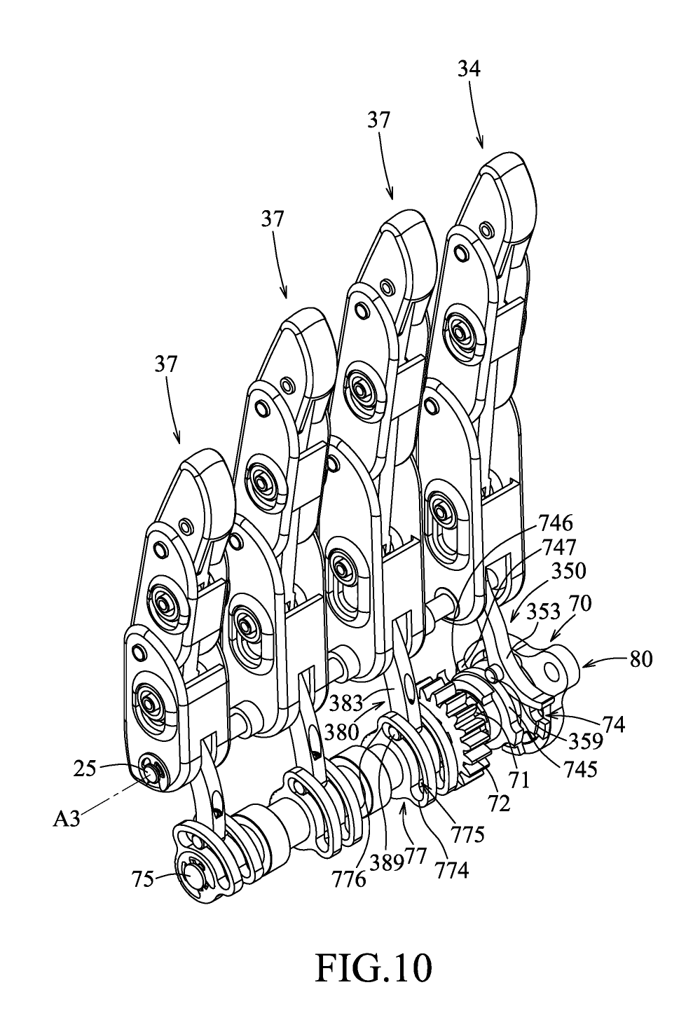

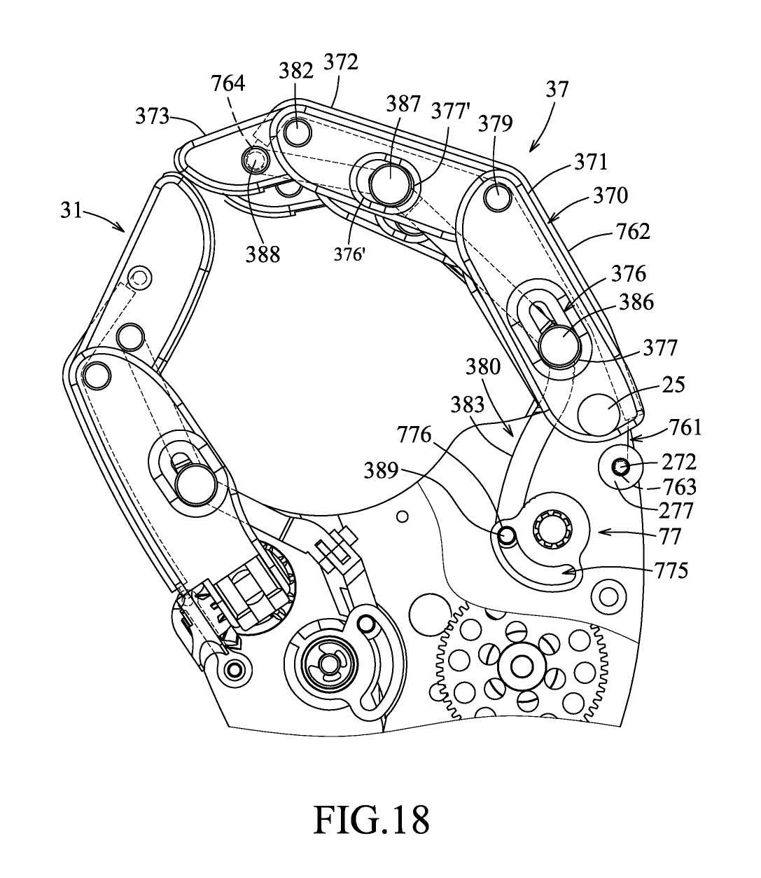

Referring to FIGS. 4, 10 and 11, the axial rod 25 of the main frame 2 defines a third axis (A3, see FIG. 10) that is parallel to the first axis (A1) (see FIG. 7). The second transmission mechanism 70 is disposed on the main frame 2, and is coupled to the drive mechanism 40 (see FIG. 5), the second finger 34 and the third fingers 37. The drive mechanism 40 is operable to drive the second transmission mechanism 70, so as to rotate the second finger 34 about the third axis (A3) between the second closed position (see FIG. 16) and the second open position (see FIGS. 2 and 23), and to rotate each of the third fingers 37 about the third axis (A3) between the third closed position (see FIGS. 16 and 18) and the third open position (see FIGS. 2 and 27). The second transmission mechanism 70 includes a second transmission shaft 71, a second transmission gear 72, a second bias assembly 73 and a second constraint member 74. The second transmission shaft 71 is rotatably mounted to a shaft hole 217 of the first side plate 21, and has an outer spline. The second transmission shaft 71 has two opposite end portions that respectively project out of the outer surface 211 and the inner surface 212 of the first side plate 21, and is formed with a communication hole 711 that extends through the opposite end portions thereof. The second transmission gear 72 has an inner spline 721. The second transmission gear 72 is sleeved on the second transmission shaft 71, and is co-rotatable with the second transmission shaft 71 by virtue of engagement between the inner spline 721 thereof and the outer spline of the second transmission shaft 71. The second transmission gear 72 is located at the inner side of the inner surface 212 of the first side plate 21, and meshes with the seventh gear 427 (see FIG. 5) of the drive mechanism 40, so that rotation of the seventh gear 427 drives rotation of the second transmission gear 72 and the second transmission shaft 71.

Referring to FIGS. 2 and 4, the second bias assembly 73 is coupled to the main frame 2 and the second finger 34 for resiliently biasing the second finger 34 to move toward the second open position. The second bias assembly 73 includes a second spring 731 and a second sheath 732. The second spring 731 is configured as an extension spring. The second sheath 732 is made of an elastic material such as rubber or silicone. The second sheath 732 is sleeved on the second spring 731, and abuts against a rear end of the second finger 34. The second spring 731 has a first connecting end 733, and a second connecting end 734 that is opposite to the first connecting end 733. The first connecting end 733 and the second connecting end 734 are respectively exposed from two opposite ends of the second sheath 732. The first connecting end 733 of the second spring 731 is connected to the insertion pin 272 of the limiting assembly 27 and is sandwiched between the jackets 276 of the second limiting member 273. The second connecting end 734 of the second spring 731 is connected to the second distal pivot axle 358 of the second finger 34. The second spring 731 resiliently biases the second finger 34 to move toward the second open position.

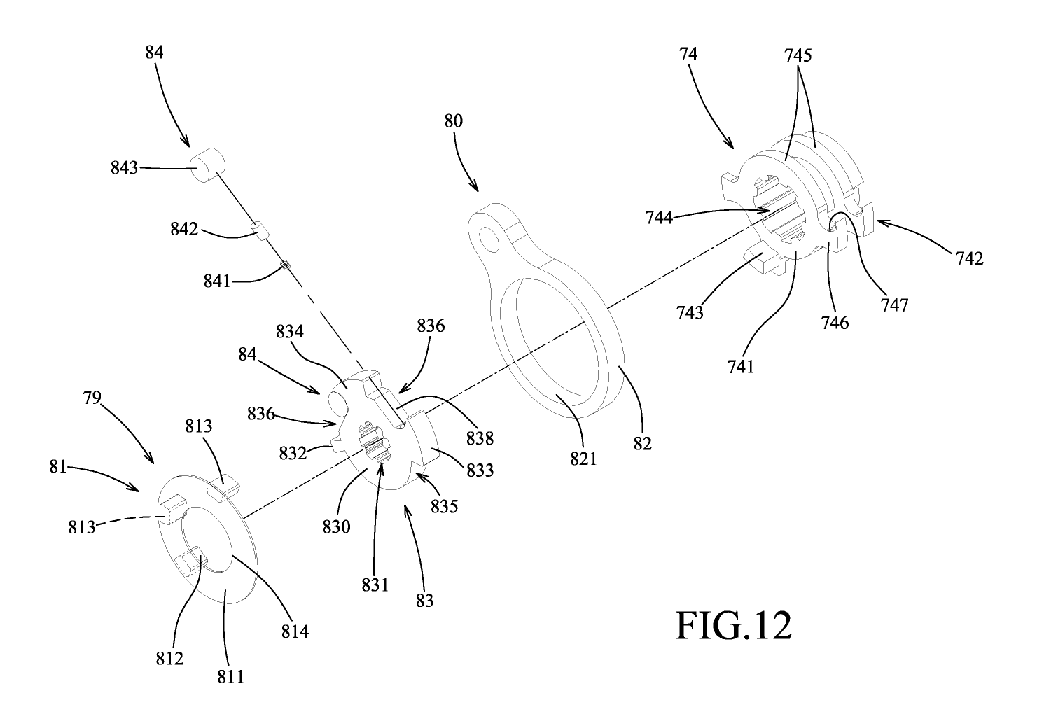

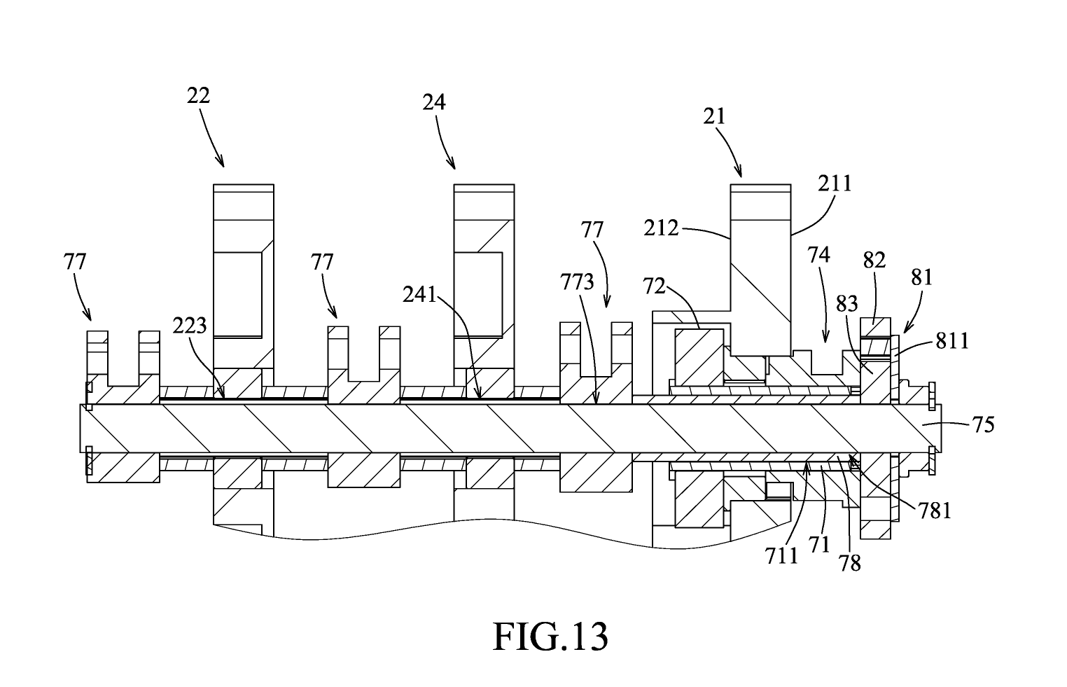

Referring to FIGS. 4, 10, 11, 12 and 13, the second constraint member 74 is sleeved on the second transmission shaft 71, is located at the outer side of the outer surface 211 of the first side plate 21, and is for constraining the second pin 359 of the second linkage 350. The second constraint member 74 has a second barrel 741, a second constraint block 742 and a poking arm 743. The second barrel 741 has an inner spline 744. The second barrel 741 is sleeved on and coupled to the second transmission shaft 71 by virtue of the engagement between the inner spline 744 thereof and the outer spline of the second transmission shaft 71, so that the second constraint member 74 is co-rotatable with the second transmission shaft 71. The second constraint block 742 is disposed on an outer surrounding surface of the second barrel 741, and includes two second limiting plates 745 that are spaced apart from each other in the axial direction of the second transmission shaft 71. The second limiting plates 745 are respectively located at the opposite lateral sides of the second proximal link 353 of the second linkage 350 for limiting wobble movement of the second proximal link 353. Each of the second limiting plates 745 has a claw portion 746 that has a second stop surface 747. The second stop surfaces 747 of the second limiting plates 745 serve to block or push the second pin 359. The poking arm 743 is disposed on an outer periphery of the second barrel 741, extends in the axial direction of the second transmission shaft 71, and is angularly spaced apart from the claw portions 746 of the second limiting plates 745. The second transmission shaft 71 is operable to rotate the second constraint member 74 for driving the second pin 359 of the second linkage 350, so that the second finger 34 can be biased by the second bias assembly 73 to move from the second closed position to the second open position, or be driven by the second constraint member 74 to move from the second open position to the second closed position via the second linkage 350.

Referring to FIGS. 4, 11 and 13, the second transmission mechanism 70 further includes a third transmission shaft 75 that is for driving the third fingers 37, a plurality of third bias assemblies 76 that respectively correspond to the third fingers 37, a plurality of third constraint members 77 that respectively correspond to the third fingers 37, a third bushing 78 that corresponds to the third transmission shaft 75, and a transmission assembly 79.

The third transmission shaft 75 has an outer spline, and rotatably extends through the communication hole 711 of the second transmission shaft 71, a shaft hole 241 of the partition plate 24 and a shaft hole 223 of the second side plate 22. The third bushing 78 has an inner spline 781. An outer surrounding surface of the third bushing 78 is smooth. The third bushing 78 is co-rotatably sleeved on the third transmission shaft 75 by virtue of the inner spline 781 thereof and the outer spline of the third transmission shaft 75, and is rotatably mounted in the communication hole 711 of the second transmission shaft 71, so that the third transmission shaft 75 is smoothly rotatable relative to the second transmission shaft 71.

Referring to FIGS. 2 and 4, the number of the third bias assemblies 76 is three. Each of the third bias assemblies 76 is coupled to the main frame 2 and a corresponding one of the third fingers 37 for resiliently biasing the corresponding third finger 37 to move toward the third open position. For the sake of brevity, only one of the third bias assemblies 76 is described in detail. The third bias assembly 76 includes a third spring 761 and a third sheath 762. The third spring 761 is configured as an extension spring. The third sheath 762 is made of an elastic material such as rubber or silicone. The third sheath 762 is sleeved on the third spring 761, and abuts against a rear end of the corresponding third finger 37. The third spring 761 has a first connecting end 763, and a second connecting end 764 that is opposite to the first connecting end 763. The first connecting end 763 and the second connecting end 764 are respectively exposed from two opposite ends of the third sheath 762. The first connecting end 763 of the third spring 761 is connected to the insertion pin 272 of the limiting assembly 27 and is sandwiched between the jackets 277 of the corresponding third limiting member 274. The second connecting end 764 of the third spring 761 is connected to the third distal pivot axle 388 of the corresponding third finger 37. The third spring 761 resiliently biases the corresponding third finger 37 to move toward the third open position.

Referring to FIGS. 10, 11 and 13, the number of the third constraint members 77 is the same as the number of the second fingers 37. In this embodiment, there are three third constraint members 77. The third constraint members 77 are co-rotatably sleeved on the third transmission shaft 75. One of the third constraint members 77 is located between the second transmission gear 72 and the partition plate 24. Another one of the third constraint members 77 is located between the second side plate 22 and the partition plate 24. The other one of the third constraint members 77 is located at the outer side of the second side plate 22. Each of the third constraint members 77 is for constraining the third pin 389 of the third linkage 380 of the corresponding third finger 37.

For the sake of brevity, only one of the third constraint members 77 is described in detail.

The third constraint member 77 has a third barrel 771 and a third constraint block 772. The third barrel 771 has an inner spline 773. The third barrel 771 is sleeved on and coupled to the third transmission shaft 75 by virtue of the engagement between the inner spline 773 thereof and the outer spline of the third transmission shaft 75, so that the third constraint member 77 is co-rotatable with the third transmission shaft 75. The third constraint block 772 is disposed on an outer surrounding surface of the third barrel 771, and includes two third limiting plates 774 that are spaced apart from each other in the axial direction of the third transmission shaft 75. The third limiting plates 774 are respectively located at the opposite lateral sides of the third proximal link 383 of the third linkage 380 of the corresponding third finger 37 for limiting wobble movement of the third proximal link 383. Each of the third limiting plates 774 is formed with an arc-shaped guide groove 775. The third pin 389 of the third linkage 380 of the corresponding third finger 37 has two opposite ends respectively engaging the guide grooves 775 of the third limiting plates 774, and is movable along the guide grooves 775. Each of the third limiting plates 774 has a third stop surface 776 that is located at an end of the guide groove 775 thereof, and an end surface 777 that is located at the other end of the guide groove 775 thereof. The third stop surfaces 776 of the third limiting plates 774 serve to block or push the third pin 389 of the third linkage 380 of the corresponding third finger 37. The third transmission shaft 75 is operable to rotate the third constraint member 77 for driving the third pin 389 of the third linkage 380 of the corresponding third finger 37, so that the corresponding third finger 37 can be biased by the corresponding third bias assembly 76 (see FIG. 4) to move from the third closed position to the third open position, or be driven by the third constraint member 77 to move from the third open position to the third closed position via the third linkage 380 thereof.

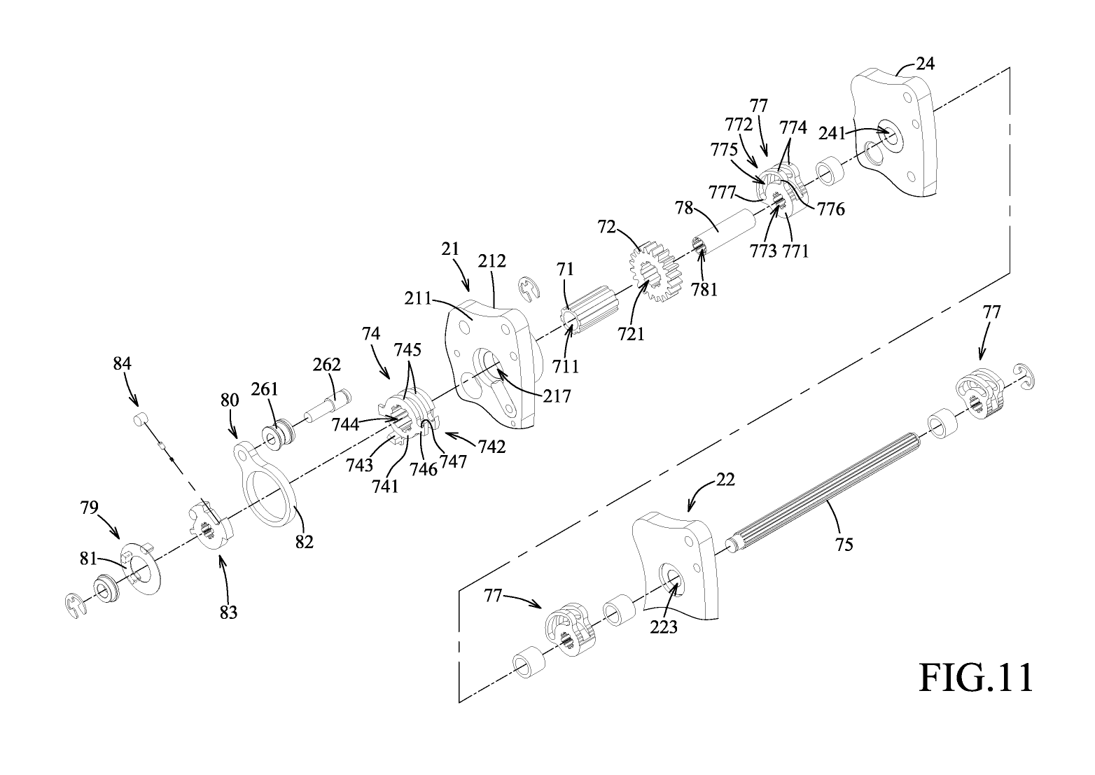

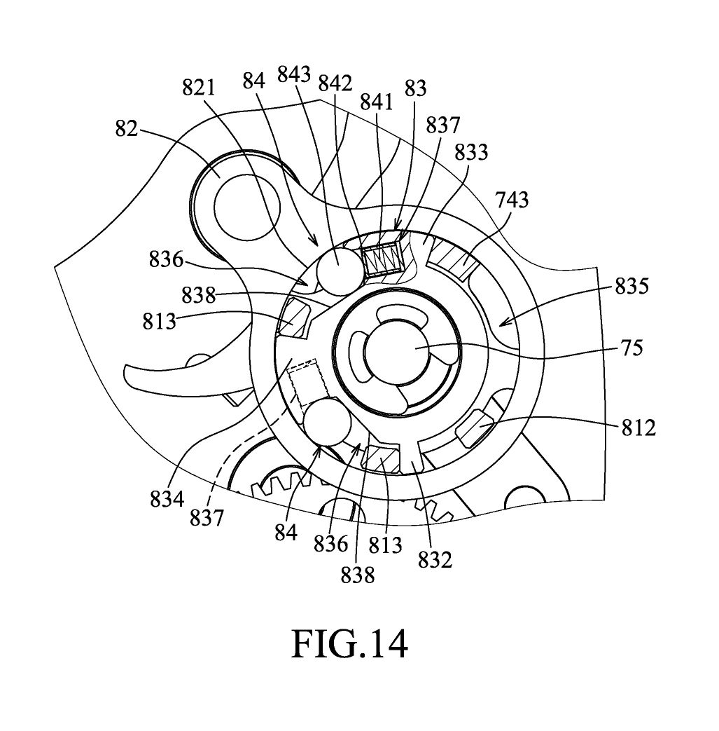

Referring to FIGS. 11, 12, 13 and 14, the transmission assembly 79 is disposed on the third transmission shaft 75, is located at one side of the second constraint member 74 opposite to the first side plate 21, and includes a clutch 80 and a release member 81. The clutch 80 includes an outer ring 82, an inner ring 83 that is disposed in the outer ring 82, and a plurality of locking modules 84 each of which is disposed between the outer ring 82 and the inner ring 83. The positioning pin 262 of the blocking assembly 26 extends through the bearing 261 and the outer ring 82 for fixing the outer ring 82 at the outer side of the first side plate 21. The bearing 261 of the blocking assembly 26 abuts against the outer ring 82, and is located between the outer ring 82 and the first side plate 21. The outer ring 82 has an inner surrounding surface 821. The inner ring 83 is surrounded by the inner surrounding surface 821 of the outer ring 82, and has a ring portion 830 that has an inner spline 831. The inner ring 83 is sleeved on and co-rotatable with the third transmission shaft 75 by virtue of the inner spline 831 thereof and the outer spline of the third transmission shaft 75. The inner ring 83 further has a first protrusion 832, a second protrusion 833 and a third protrusion 834 that are disposed on an outer surrounding surface of the ring portion 830, and that are angularly spaced apart from one another. The inner ring 83 further has an arc-shaped retaining groove 835 that is defined between the first protrusion 832 and the second protrusion 833, two roller grooves 836 that are respectively defined between the first protrusion 832 and the third protrusion 834 and between the second protrusion 833 and the third protrusion 834, and two abutment surfaces 838 that respectively and partially define the roller grooves 836. Each of the second protrusion 833 and the third protrusion 834 is formed with a mounting groove 837 (see FIG. 14) that is in spatial communication with a respective one of the roller grooves 836. The number of the locking modules 84 is two. Each of the locking modules 84 includes a compression spring 841, a spring sheath 842 that is sleeved on the compression spring 841, and a roller 843. The compression spring 841 and the spring sheath 842 of each of the locking modules 84 are mounted in the mounting groove 837 of a respective one of the second protrusion 833 and the third protrusion 834. The roller 843 of each of the locking modules 84 is movably disposed in a respective one of the roller grooves 836. The compression spring 841 of each of the locking modules 84 resiliently biases the corresponding spring sheath 842 to push the corresponding roller 843 for maintaining the corresponding roller 843 at a locking position where the corresponding roller 843 is pushed against the inner surrounding surface 821 of the outer ring 82 and the corresponding abutment surface 838 so as to permit the inner ring 83 to rotate relative to the outer ring 82 in only one direction (i.e., each of the locking modules 84 is in a locking state).

The release member 81 includes a cover plate 811, a first projection 812 and a plurality of second projections 813. The cover plate 811 is formed with a pivoting hole 814 that permits the third transmission shaft 75 to rotatably extend therethrough, and covers the inner ring 83 and a portion of the outer ring 82. The first projection 812 and the second projections 813 project from an inner surface of the cover plate 811, and are angularly spaced apart from each other. The number of the second projections 813 is two. The first projection 812 of the release member 81 is movably received in the arc-shaped retaining groove 835 of the inner ring 83 for being pushed by the poking arm 743 of the second constraint member 74. Each of the second projections 813 is movably received in a respective one of the roller grooves 836 for pushing the corresponding roller 843 against the biasing action of the corresponding compression spring 841 so as to move the corresponding roller 843 to an unlocking position where the corresponding roller 843 is separated from at least one of the inner surrounding surface 821 of the outer ring 82 and the corresponding abutment surface 838 so that the inner ring 83 is freely rotatable relative to the outer ring 82 (i.e., each of the locking modules 84 is in an unlocking state).

The poking arm 743 of the second constraint member 74 extends into the arc-shaped retaining groove 835 of the inner ring 83, and is located between the second protrusion 833 of the inner ring 83 and the first projection 812 of the release member 81. In this embodiment, the poking arm 743 of the second constraint member 74 has an arc-shaped cross-section that has an arc length smaller than the arc length of the arc-shaped retaining groove 835 of the inner ring 83. The poking arm 743 of the second constraint member 74 is rotatable in a direction for pushing the first projection 812 of the release member 81 to rotate the release member 81, so that the second projections 813 of the release member 81 respectively push the rollers 843 of the locking modules 84 to the unlocking position, and that the second projections 813 of the release member 81 respectively push the second protrusion 833 and the third protrusion 834 of the inner ring 83 via the rollers 843 to rotate the inner ring 83, the third transmission shaft 75 and the third constraint members 77, so as to permit the third pins 389 of the third fingers 37 to be moved by the third bias assemblies 76. The poking arm 743 of the second constraint member 74 is also rotatable in an opposite direction for pushing the second protrusion 833 of the inner ring 83 to rotate the inner ring 83, the third transmission shaft 75 and the third constraint members 77, so that the third stop surfaces 776 of each of the third constraint members 77 push and move the third pin 389 of the corresponding third finger 37.

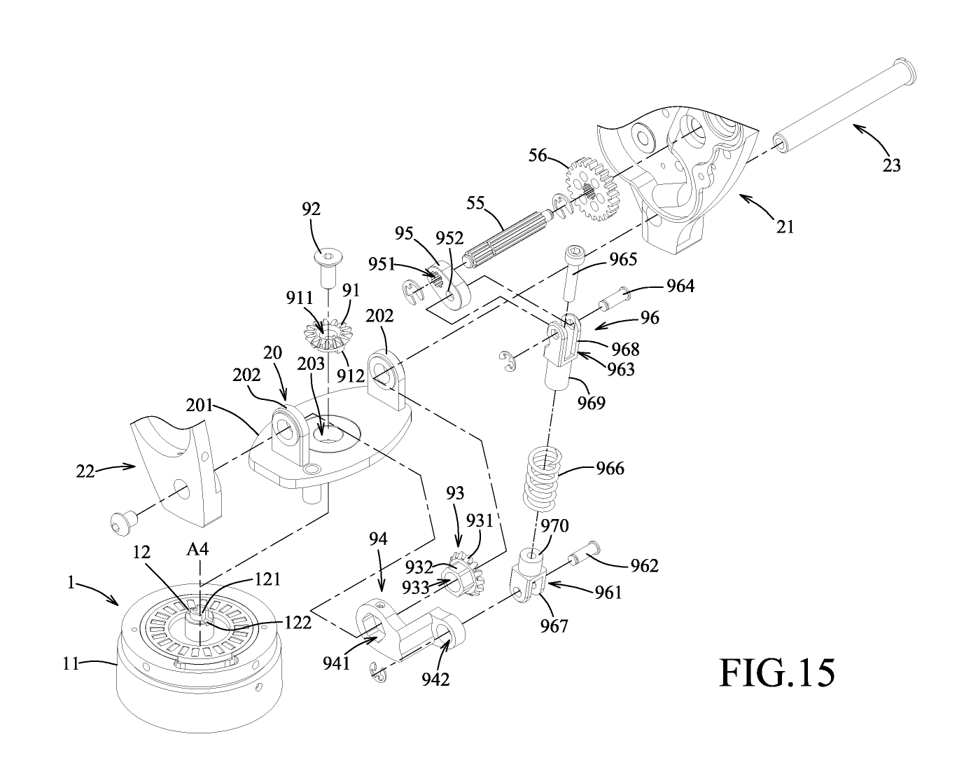

Referring to FIGS. 1, 3 and 15, the wrist frame 1 includes a frame body 11, and a central axle 12 that is disposed on a top end of the frame body 11. The central axle 12 is perpendicular to the axial post 23 of the main frame 2, and defines a fourth axis (A4, see FIG. 15) that is orthogonal to the first axis (A1) (see FIGS. 7 and 8). The central axle 12 is formed with a threaded hole 121, and two diametrically-opposite slots 122 that are in spatial communication with the threaded hole 121. The base plate portion 201 of the support plate 20 of the main frame 2 is formed with a pivoting hole 203. The central axle 12 rotatably extends through the base plate portion 201 of the support plate 20 via the pivoting hole 203, and projects out of a top surface of the base plate portion 201.

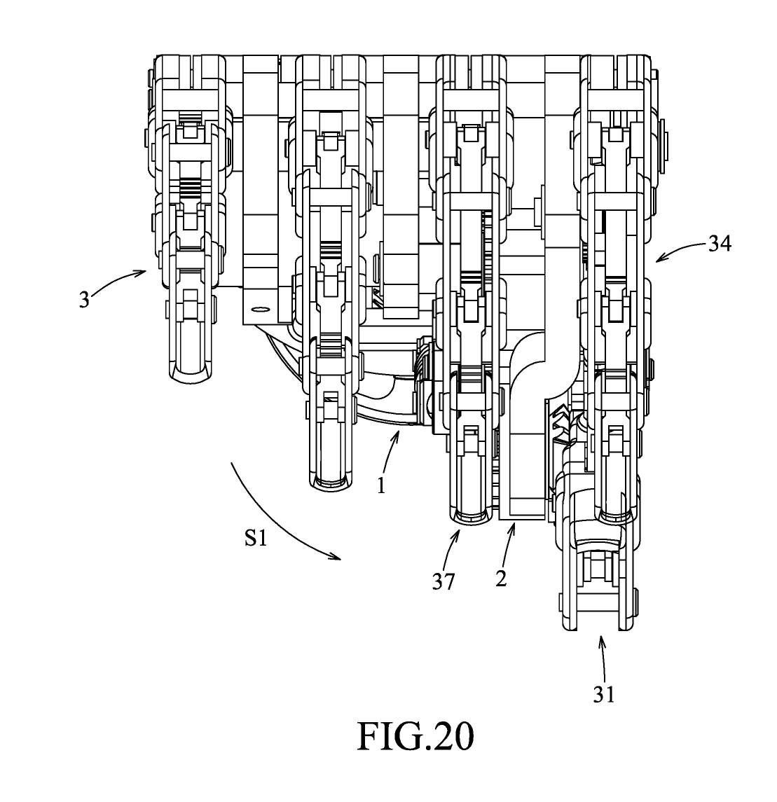

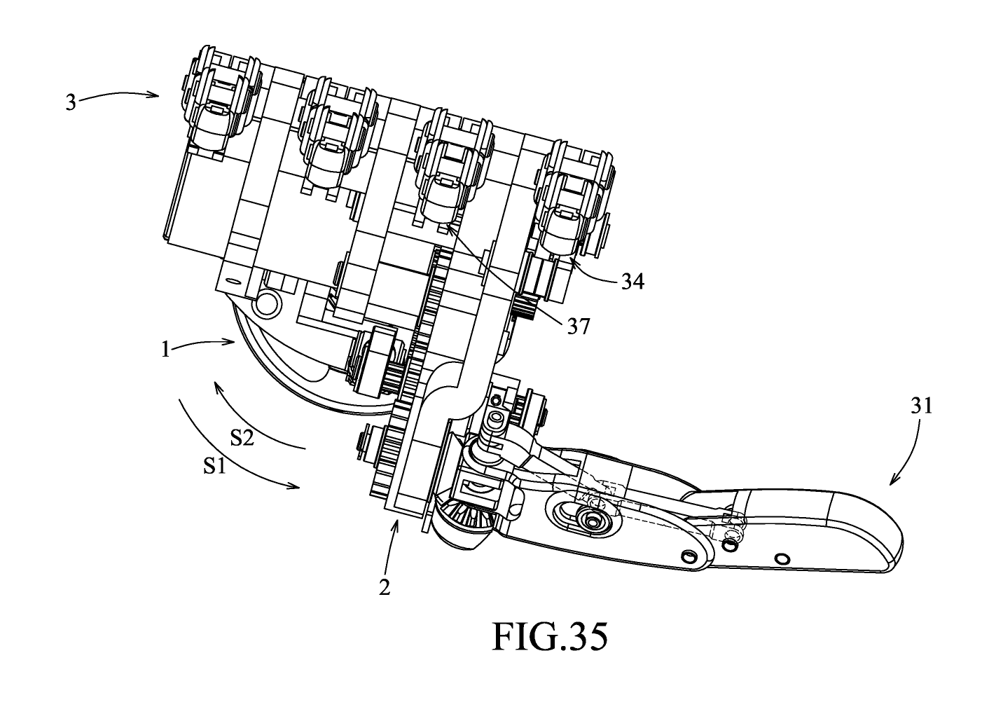

The third transmission mechanism 90 is coupled to the first transmission shaft 55 of the first transmission mechanism 50, the axial post 23 of the main frame 2 and the central axle 12 of the wrist frame 1. The first transmission mechanism 50 and the third transmission mechanism 90 is driven by the drive mechanism 40 to rotate the main frame 2 relative to the wrist frame 1 about the fourth axis (A4) between a first twist position (see FIG. 20) and a second twist position (see FIG. 35). The third transmission mechanism 90 includes a fixed gear 91, a screw 92, a movable gear 93, a first swing arm 94, a second swing arm 95 and a link assembly 96. The fixed gear 91 is formed with a through hole 911 that permits the central axle 12 of the wrist frame 1 to extend therethrough. The fixed gear 91 has two diametrically-opposite engaging blocks 912 (only one is visible in FIG. 15) at a bottom end thereof that respectively engage the slots 122 of the central axle 12, so that the fixed gear 91 is not rotatable relative to the central axle 12. The screw 92 extends through the through hole 911 of the fixed gear 91, engages threadedly the threaded hole 121 of the central axle 12, and has a head portion abutting against the fixed gear 91, so as to prevent separation of the fixed gear 91 from the central axle 12.

The movable gear 93 is rotatably sleeved on the axial post 23 of the main frame 2, and has a gear portion 931 and a hollow axle portion 932 that are coaxially-arranged. The gear portion 931 and the hollow axle portion 932 of the movable gear 93 cooperatively define a pivoting hole 933 that permits the axial post 23 of the main frame 2 to rotatably extend therethrough. The gear portion 931 of the movable gear 93 meshes with the fixed gear 91. The hollow axle portion 932 of the movable gear 93 has a non-circular cross-section. The first swing arm 94 has an engaging end portion formed with an engaging hole 941, and a pivoting end portion formed with a pivoting hole 942. The engaging hole 941 of the first swing arm 94 is complementary to the hollow axle portion 932 of the :movable gear 93, and is engaged with the hollow axle portion 932 of the movable gear 93, so that the first swing arm 94 is co-rotatable with the movable gear 93. The second swing arm 95 has an engaging end portion formed with an inner spline 951, and a pivoting end portion formed with a pivoting hole 952. The second swing arm 95 is sleeved on and co-rotatable with the first transmission shaft 55 of the first transmission mechanism 50 by virtue of the engagement between the inner spline 951 thereof and the outer spline of the first transmission shaft 55.

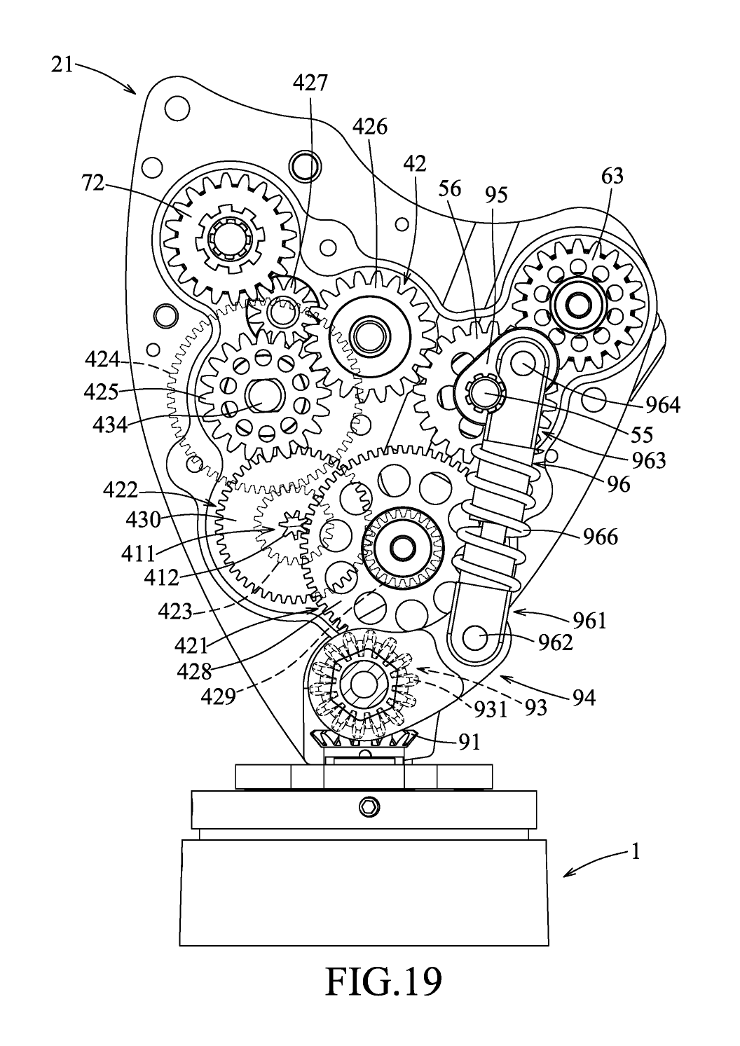

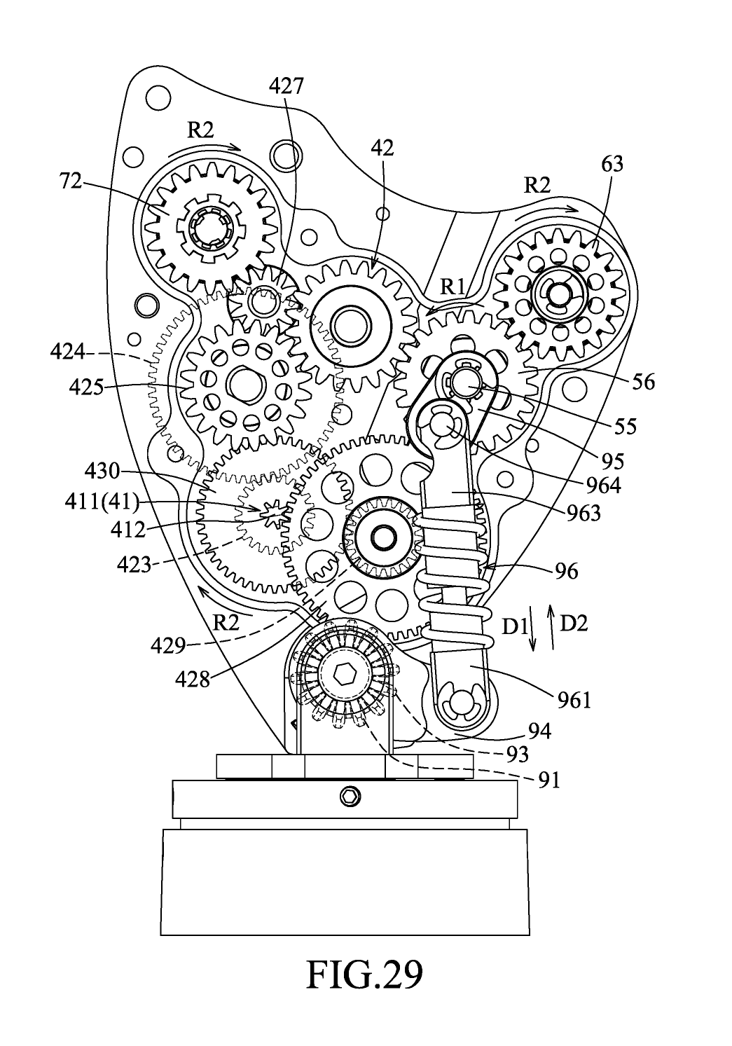

The link assembly 96 has two opposite end portions respectively and pivotally connected to the pivoting end portion of the first swing arm 94 and the pivoting end portion of the second swing arm 95. Rotation of the first transmission shaft 55 and the second swing arm 95 moves the link assembly 96 between a first move position (see FIG. 19) and a second move position (see FIG. 29) to rotate the first swing arm 94 and the movable gear 93 relative to the main frame 2, so as to rotate the main frame 2 relative to the wrist frame 1 about the fourth axis (A4) between the first twist position (see FIG. 20) and the second twist position (see FIG. 35) by virtue of the meshing between the movable gear 93 and the fixed gear 91.

Specifically, the link assembly 96 of this embodiment includes a first link 961, a first connecting rod 962. a second link 963, a second connecting rod 964, a screw 965 and a compression spring 966. The first link 961 has a bifurcated portion 967 that brackets the pivoting end portion of the first swing arm 94. The first connecting rod 962 extends through the pivoting hole 942 of the first awing arm 94 to pivotally interconnect the bifurcated portion 967 of the first link 961 and the pivoting end portion of the first swing arm 94. The second link 963 has a bifurcated portion 968 that brackets the pivoting end portion of the second swing arm 95. The second connecting rod 964 extends through the pivoting hole 952 of the second awing arm 95 to pivotally interconnect the bifurcated portion 968 of the second link 963 and the pivoting end portion of the second swing arm 95. The screw 965 extends through a hollow portion 969 of the second link 963, and engages threadedly an internally-threaded hollow portion 970 of the first link 961 so as to interconnect the first link 961 and the second link 963. The compression spring 966 is sleeved on the hollow portion 970 of the first link 961 and the hollow portion 969 of the second link 963, and has two opposite ends respectively abutting against the bifurcated portion 967 of the first link 961 and the bifurcated portion 968 of the second link 963.

The hold unit 3 of the prosthetic hand 100 of this disclosure is operable to switch among a closed state (see FIG. 16), a pointing state (see FIG. 23), an open state (see FIG. 25) and an eversion state (see FIG. 30).

Referring to FIG. 16, when the hold unit 3 of the prosthetic hand 100 is in the closed state, the first finger 31 is at the first closed position, the second finger 34 is at the second closed position, each of the third fingers 37 is at the third closed position, and the main frame 2 is at the first twist position (see FIG. 20) relative to the wrist frame 1. The first constraint member 57 of the constraint assembly 54 of the first transmission mechanism 50 is at a first rotational position (see FIG. 16) where the first stop surfaces 577 of the first limiting plates 575 of the first constraint member 57 block the first pin 329 of the first linkage 320 of the first finger 31. At this time, the first proximal pivot axle 326 of the first linkage 320 is spaced apart from the second pivot axle 516 of the hinge 51 of the first transmission mechanism 50, and is not coaxial with the second pivot axle 516. The first intermediate pivot axle 327 of the first linkage 320 is at the proximal ends 321 of the first oblong grooves 319 of the first proximal phalange 311, and the first distal pivot axle 328 of the first linkage 320 is located behind and above the pivoting axle 315 of the first finger 31. Since the first intermediate pivot axle 327 and the first distal pivot axle 328 of the first linkage 320 are respectively connected to the first proximal phalange 311 and the first distal phalange 312 of the first finger body 310, the first distal phalange 312 is bent rearwardly relative to the first proximal phalange 311, and the first distal end portion 314 of the first finger body 310 is proximate to the second finger 34 and the first end surface 210 of the first side plate 21. By virtue of the connection between the first linkage 320 and the first finger body 310, when the first constraint member 57 is at the first rotational position so that the first stop surfaces 577 of the first limiting plates 575 of the first constraint member 57 block the first pin 329 of the first linkage 320, the first finger 31 is bent, and is maintained at the first closed position.

Since the first connecting end 533 and the second connecting end 534 of the first spring 531 of the first bias assembly 53 are respectively connected to the support arm 29 and the pin member 330 of the first finger 31, and since the first sheath 532 of the first bias assembly 53 abuts against a front end of the first finger 31 (e.g., the pivoting axle 315), the first spring 531 is deformed to generate a restoring force when the first finger 31 is at the first closed position.

In addition, when the hold unit 3 is in the closed state and when the first constraint member 57 of the constraint assembly 54 of the first transmission mechanism 50 is at a first rotational position, the poking arm 582 of the poking member 58 of the constraint assembly 54 abuts against the first constraint block 573 of the first constraint member 57. Since the arc length of the poking arm 582 is smaller than the arc length of the arc-shaped groove 579 of the first constraint member 57, the poking arm 582 of the poking member 58 is spaced apart from the first protruding block 572 of the first constraint member 57.

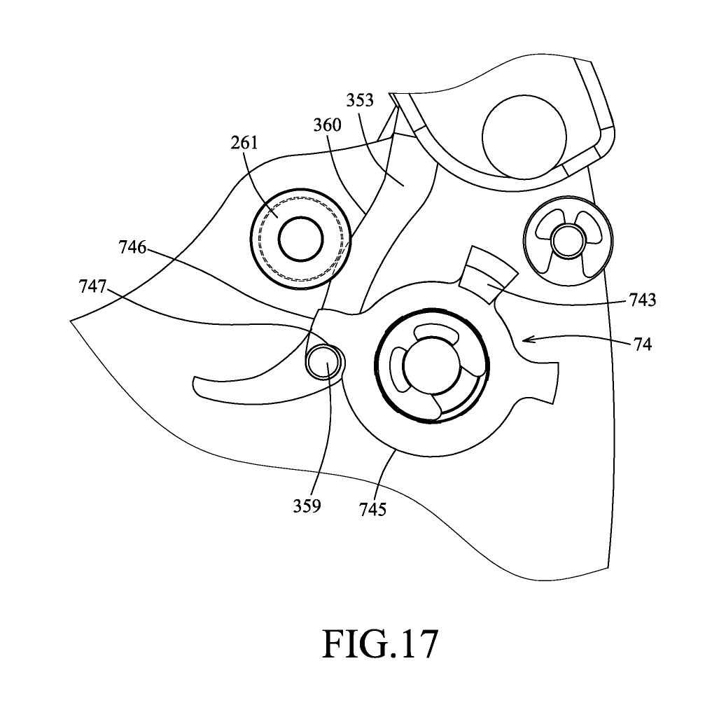

Referring to FIGS. 16 and 17, on the other hand, the second constraint member 74 of the second transmission mechanism 70 is at a first pivoting position (see FIG. 17) where the second stop surfaces 747 of the second limiting plates 745 of the second constraint member 74 block the second pin 359 of the second linkage 350 of the second finger 34. At this time, the front surface 360 of the second proximal link 353 of the second linkage 350 abuts against the bearing 261 of the blocking module 26, the second proximal pivot axle 356 of the second linkage 350 is located at the proximal ends 347 of the second oblong grooves 346 of the second proximal phalange 341, and the second intermediate pivot axle 357 of the second linkage 350 is located at the proximal ends 347′ of the second oblong grooves 346′ of the second intermediate phalange 342. The second intermediate pivot axle 357 is located in front of and above the second proximal pivot axle 356. The second distal pivot axle 358 is located in front of the second intermediate pivot axle 357. Since the second proximal pivot axle 356, the second intermediate pivot axle 357 and the second distal pivot axle 358 of the second linkage 350 are respectively connected to the second proximal phalange 341, the second intermediate phalange 342 and the second distal phalange 343 of the second finger body 340, the second proximal phalange 341 is bent forwardly relative to the main frame 2, the second intermediate phalange 342 is bent forwardly relative to the second proximal phalange 341, the second distal phalange 343 is bent forwardly relative to the second intermediate phalange 342, and the second distal end portion 351 of the second finger body 340 is in contact with the first distal end portion 314 of the first finger body 310. By virtue of the connection between the second linkage 350 and the second finger body 340, when the second constraint member 74 is at the first pivoting position so that the second stop surfaces 747 of the second limiting plates 745 of the second constraint member 74 block the second pin 359 of the second linkage 350, the second finger 34 is bent, and is maintained at the second closed position.

Since the first connecting end 733 and the second connecting end 734 of the second spring 731 of the second bias assembly 73 are respectively connected to the insertion pin 272 of the limiting assembly 27 and the second distal pivot axle 358 of the second finger 34, and since the second sheath 732 of the second bias assembly 73 abuts against a rear end of the second finger 34 (e.g., the pivoting axle 349 and the pivoting axle 352), the second spring 731 is deformed to generate a restoring force when the second finger 34 is at the second closed position.

In addition, when the second constraint member 74 is at the first pivoting position, the inner ring 83 of the clutch 80 of the transmission assembly 79 is at a first angular position (see FIG. 16). At this time, the roller 843 of each of the locking modules 84 is at the locking position, the first projection 812 of the release member 81 of the transmission assembly 79 is adjacent to the first protrusion 832 of the inner ring 83, and the poking arm 743 of the second constraint member 74 abuts against the second protrusion 833 of the inner ring 83. Since the arc length of the poking arm 743 of the second constraint member 74 is smaller than the arc length of the arc-shaped retaining groove 835 of the inner ring 83, the poking arm 743 of the second constraint member 74 is spaced apart from the first protrusion 832 of the inner ring 83 and the first projection 812 of the release member 81.

Referring to FIGS. 16 to 18, when the second constraint member 74 is at the first pivoting position, each of the third constraint members 77 is at a first rotating position (see FIG. 18) where the third stop surfaces 776 of each of the third constraint members 77 block the third pin 389 of the third linkage 380 of the corresponding third finger 37. At this time, for each of the third fingers 37, the third proximal pivot axle 386 of the third linkage 380 is located at the proximal ends 377 of the third oblong grooves 376 of the third proximal phalange 371, and the third intermediate pivot axle 387 of the third linkage 380 is located at the proximal ends 377′ of the third oblong grooves 376′ of the third intermediate phalange 372. The third intermediate pivot axle 387 is located in front of and above the third proximal pivot axle 386. The third distal pivot axle 388 is located in front of the third intermediate pivot axle 387. Since the third proximal pivot axle 386, the third intermediate pivot axle 387 and the third distal pivot axle 388 of the third linkage 380 are respectively connected to the third proximal phalange 371, the third intermediate phalange 372 and the third distal phalange 373 of the third finger body 370, the third proximal phalange 371 is bent forwardly relative to the main frame 2, the third intermediate phalange 372 is bent forwardly relative to the third proximal phalange 371, and the third distal phalange 373 is bent forwardly relative to the third intermediate phalange 372. Each of the third fingers 37 is proximate to the first finger 31. By virtue of the connection between the third linkage 380 and the third finger body 370 of each of the third fingers 37, when each of the third constraint members 77 is at the first rotating position so that the third stop surfaces 776 of each of the third constraint members 77 block the third pin 389 of the corresponding third finger 37, the corresponding third finger 37 is bent, and is maintained at the third closed position.

Since the first connecting end 763 and the second connecting end 764 of the third spring 761 of each of the third bias assemblies 76 are respectively connected to the insertion pin 272 of the limiting assembly 27 and the third distal pivot axle 388 of the corresponding third finger 37, and since the third sheath 762 of the third bias assembly 76 abuts against the rear end of the corresponding third finger 37 (e.g., the pivoting axle 379 and the pivoting axle 382), the third spring 761 is deformed to generate a restoring force when the corresponding third finger 37 is at the third closed position.

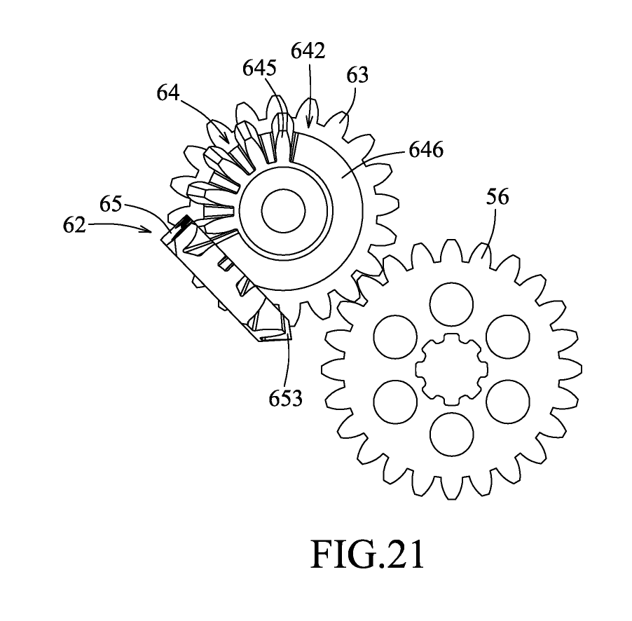

Referring to FIGS. 16, 19, 20 and 21, when the hold unit 3 is in the closed state and when first constraint member 57 of the first transmission mechanism 50 is at the first rotational position, the link assembly 96 is at the first move position. At this time, the second connecting rod 964 of the link assembly 96 is higher than the first transmission shaft 55, the main frame 2 is at the first twist position (see FIG. 20) relative to the wrist frame 1, and the intermittent gear set 62 is in the non-meshing state (see FIG. 21) in which the toothed sector 645 of the active gear 64 is separated from the annular gear section 653 of the passive gear 65.

The following paragraphs describe the switch of the hold unit 3 from the closed state (see FIG. 16) to the pointing state (see FIG. 23) where the first finger 31 is at the first closed position, where the second finger 34 is at the second open position, and where each of the third fingers 37 is at the third closed position.

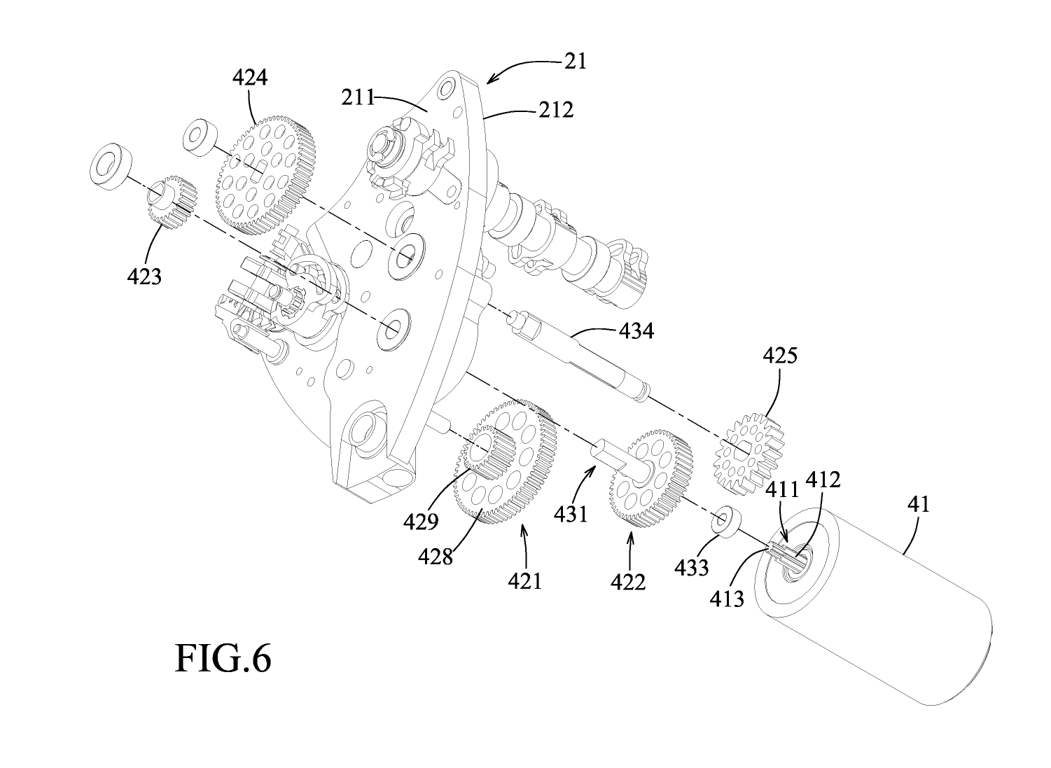

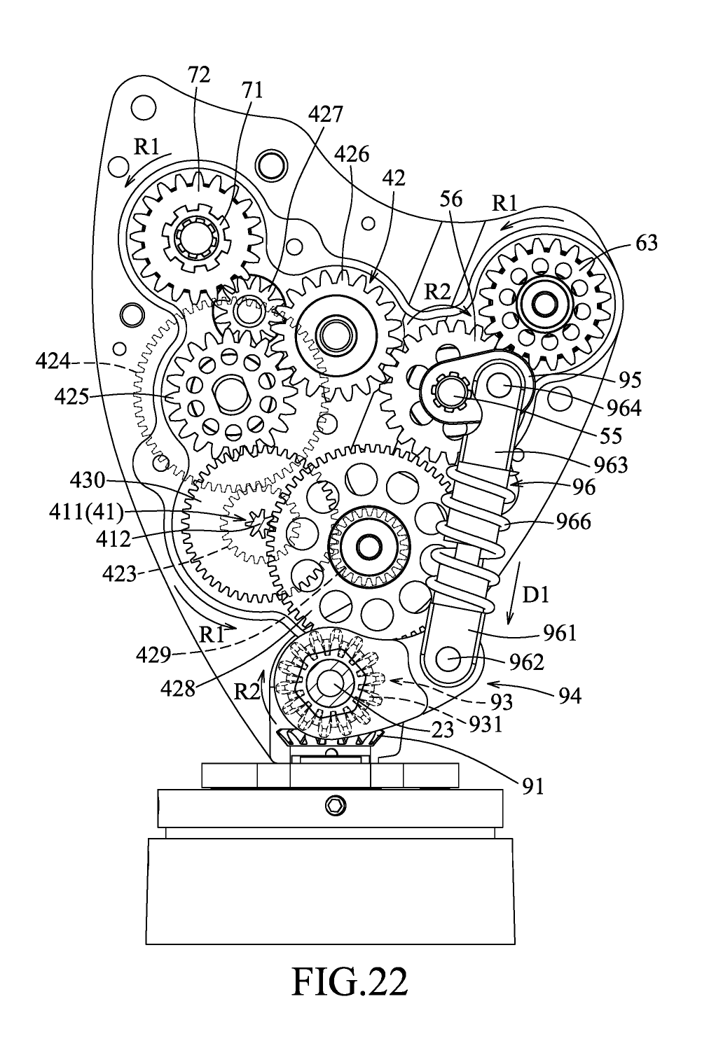

Referring to FIG. 22, the user contracts the muscle of the upper limb to generate an electromyography signal. The first sensor controls the output shaft 411 of the drive motor 41 (see FIG. 6) to rotate in a first rotational direction (R1) upon receipt of the electromyography signal, so as to drive rotation of the first transmission gear 56 of the first transmission mechanism 50 in a second rotational direction (R2) opposite to the first rotational direction (R1) via the reduction gear train 42, and to drive rotation of the second transmission gear 72 of the second transmission mechanism 70 in the first rotational direction (R1).

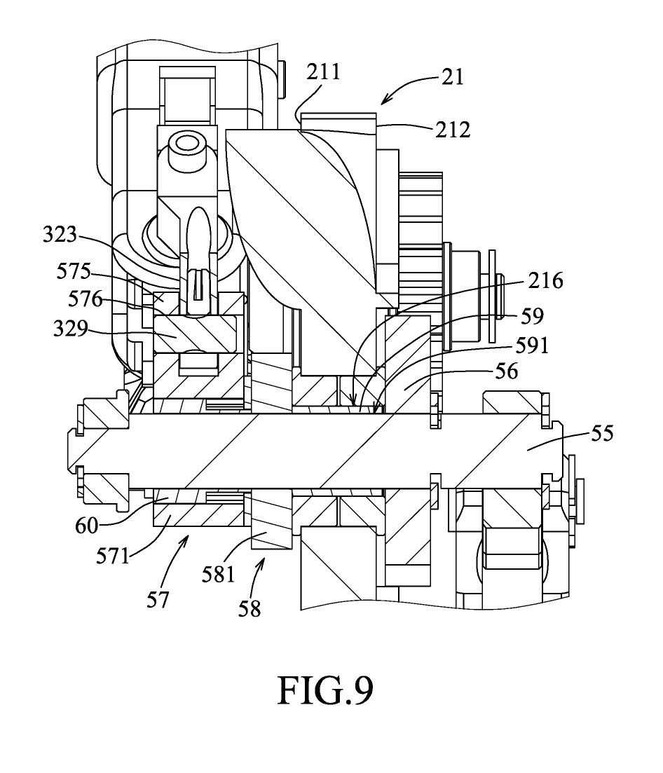

Referring to FIGS. 9, 22 and 23, the rotation of the first transmission gear 56 in the second rotational direction (R2) drives synchronous rotation of the poking member 58 in the second rotational direction (R2) via the first transmission shaft 55. Since the first constraint member 57 is rotatable sleeved on the first transmission shaft 55 via the second bushing 60, the poking member 58 is rotatable relative to the first constraint member 57. Since the poking arm 582 of the poking member 58 is initially spaced apart from the first protruding block 572 of the first constraint member 57, the rotation of the poking member 58 in the second rotational direction (R2) does not drive rotation of the first constraint member 57 until the poking arm 582 of the poking member 58 is in contact with the first protruding block 572, as shown in FIG. 23. At this time, the poking arm 582 of the poking member 58 is spaced apart from the first constraint block 573 of the first constraint member 57, the first constraint member 57 is maintained at the first rotational position, and the first finger 31 is maintained at the first closed position.

Referring to FIGS. 22, 23 and 24 on the other hand, the rotation of the second transmission gear 72 of the second transmission mechanism 70 in the first rotational direction (R1) drives rotation of the second constraint member 74 in the first rotational direction (R1) via the second transmission shaft 71. During the rotation of the second constraint member 74 in the first rotational direction (R1), the second stop surfaces 747 of the claw portions 746 of the second limiting plates 745 rotate to permit movement of the second pin 359 of the second finger 34. The restoring force generated by the second spring 731 of the second bias assembly 73 moves the second distal pivot axle 358 of the second finger 34 upwardly and rearwardly, so that the second distal phalange 343 is rotated about the pivoting axle 352 relative to the second intermediate phalange 342 in the first rotational direction (R1). At the same time, the second distal pivot axle 358 pulls the second distal link 355 so that the second intermediate pivot axle 357 of the second linkage 350 moves toward the distal ends 348′ of the second oblong grooves 346′ of the second intermediate phalange 342, and that the second proximal pivot axle 356 of the second linkage 350 moves toward the distal ends 348 of the second oblong grooves 346 of the second proximal phalange 341. During the movement of the second intermediate pivot axle 357 toward the distal ends 348′ of the second oblong grooves 346′, the second intermediate phalange 342 is rotated about the pivoting axle 349 relative to the second proximal phalange 341 in the first rotational direction (R1). During the movement of the second proximal pivot axle 356 toward the distal ends 348 of the second oblong grooves 346, the second proximal phalange 341 is rotated about the third axis (A3) in the first rotational direction (R1), and the second pin 359 of the second linkage 350 is moved to abut against the second stop surfaces 747 of the second constraint member 74.

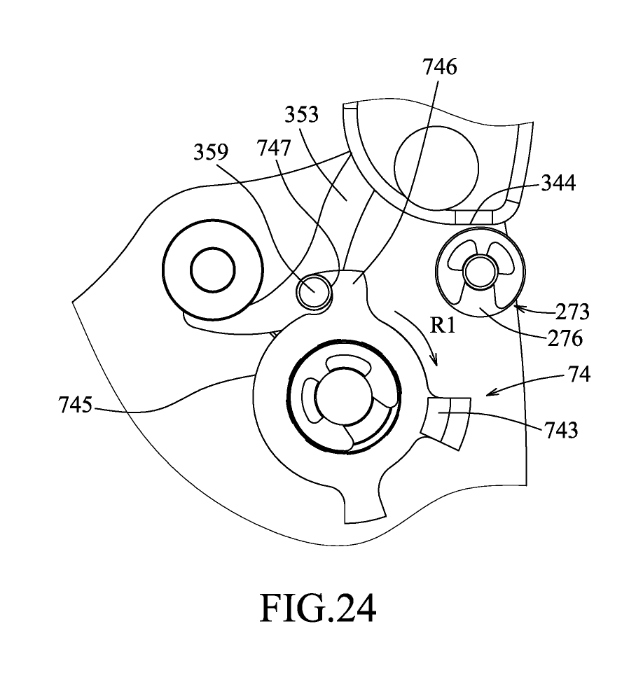

When the poking member 58 rotates the poking arm 582 to abut against the first protruding block 572 of the first constraint member 57, the second constraint member 74 is rotated to a second pivoting position, as shown in FIG. 24. At this time, the second stop surfaces 747 of the second limiting plates 745 of the second constraint member 74 still block the second pin 359 of the second linkage 350, and the poking arm 743 of the second constraint member 74 is separated from the second protrusion 833 of the inner ring 83 and abuts against the first projection 812 of the release member 81 without rotating the release member 81. Since the second intermediate pivot axle 357 of the second linkage 350 is limited within the distal ends 348′ of the second oblong grooves 346′ of the second intermediate phalange 342, since the second proximal pivot axle 356 of the second linkage 350 is limited within the distal ends 348 of the second oblong grooves 346 of the second proximal phalange 341, and since the second proximal end portion 344 of the second finger 34 is limited by the second limiting member 273 of the limiting assembly 27, the second finger 34 is stretched and steadily maintained at the second open position.

It should be noted that since the arc length of the arc-shaped groove 579 of the first constraint member 57 is greater than the arc length of the poking arm 582 of the poking member 58, rotation of the poking member 58 in the second rotational direction (R2) does not immediately rotate the first constraint member 57. As such, when the second constraint member 74 is rotated from the first pivoting position to the second pivoting position, the first constraint member 57 is maintained at the first rotational position to prevent movement of the first pin 329 of the first linkage 320, so as to maintain the first finger 31 at the first closed position. In addition, since the arc length of the arc-shaped retaining groove 835 of the inner ring 83 is greater than the arc length of the poking arm 743 of the second constraint member 74, rotation of the second constraint member 74 from the first pivoting position to the second pivoting position in the first rotational direction (R1) does not immediately rotate the release member 81, so that the rollers 843 of the locking modules 84 are maintained at the locking position so as to maintain the inner ring 83 of the clutch 80 of the transmission assembly 79 at the first angular position (see FIG. 16), and to maintain each of the third constraint members 77 at the first rotating position (see FIG. 18). Therefore, each of the third fingers 37 is maintained at the third closed position.

Referring to FIGS. 21, 22 and 23, rotation of the first transmission gear 56 in the second rotational direction (R2) rotates the connecting gear 63 of the first transmission mechanism 50 and the active gear 64 of the intermittent gear set 62 in the first rotational direction (R1). Since the intermittent gear set 62 is maintained in the non-meshing state (see FIG. 21) in which the toothed sector 645 of the active gear 64 is separated from the annular gear section 653 of the passive gear 65, the passive gear 65 is not rotated when the poking arm 582 of the poking member 58 is rotated by the first transmission shaft 55 (see FIG. 7) to contact the first protruding block 572 of the first constraint member 57.

Referring to FIGS. 20 and 22, since the upper and lower ends of the link assembly 96 are respectively pivoted to the second swing arm 95 and the first swing arm 94, rotation of the first transmission gear 56 in the second rotational direction (R2) rotates the second swing arm 95 to move the link assembly 96 downwardly in a push direction (D1) so as to rotate the first swing arm 94 and the movable gear 93 of the third transmission mechanism 90 about the axial post 23 in the second rotational direction (R2). By such, when the hold unit 3 of the prosthetic hand 100 is switched from the closed state to the pointing state, the main frame 2 is rotated relative to the wrist frame 1 in a first swivel direction (S1) by an angle by virtue of the meshing between the movable gear 93 and the fixed gear 91.

In this embodiment, the rotation of the second swing arm 95 in the second rotational direction (R2) moves the second link 963 to push the compression spring 966, and the compression spring 966 therefore pushes the first link 961 to rotate the first swing arm 94 and the movable gear 93 in the second rotational direction (R2). The compression spring 966 serves to cushion the force transmitted between the first link 961 and the second link 963 for alleviating wear of the movable gear 93 and the fixed gear 91.

According to the above, when the hold unit 3 is switched from the closed state (see FIG. 16) to the pointing state (see FIG. 23), only the second finger 34 is moved from the second closed position to the second open position. The first finger 31 is maintained at the first closed position. Each of the third fingers 37 is maintained at the third closed position. By such, the user can use the second distal end portion 351 of the second finger 34 to operate the touch screen of a smart phone, or to depress a push button.

The following paragraphs describe the switch of the hold unit 3 from the pointing state (see FIG. 23) to the open state (see FIG. 25) where the first finger 31 is at the first open position, where the second finger 34 is at the second open position, and where each of the third fingers 37 is at the third open position.

Referring to FIG. 22, the user continues to contract the muscle of the upper limb, and the first sensor controls the output shaft 411 of the drive motor 41 (see FIG. 6) to rotate in the first rotational direction (R1) upon receipt of the electromyography signal, so as to drive rotation of the first transmission near 56 of the first transmission mechanism 50 in the second rotational direction (R2), and to drive rotation of the second transmission gear 72 of the second transmission mechanism 70 in the first rotational direction (R1).

Referring to FIGS. 22 and 25, when the poking member 58 is rotated by the first transmission gear 56 in the second rotational direction (R2) via the first transmission shaft 55 after the poking arm 582 of the poking member 58 is in contact with the first protruding block 572 of the first constraint member 57, the poking arm 582 of the poking member 58 pushes the first protruding block 572 of the first constraint member 57 to rotate the first constraint member 57 in the second rotational direction (R2). During the rotation of the first constraint member 57 in the second rotational direction (R2), the first stop surfaces 577 of the first limiting plates 575 of the first constraint member 57 rotate in the second rotational direction (R2) to permit movement of the first pin 329 of the first linkage 320 of the first finger 31. The restoring force generated by the first spring 531 of the first bias assembly 53 moves the pin member 330 of the first finger body 310 upwardly and forwardly, so that the first distal phalange 312 is rotated about the pivoting axle 315 relative to the first proximal phalange 311 in the second rotational direction (R2). During the movement of the first distal phalange 312, the first distal phalange 312 drives the first proximal phalange 311, the hinge 51 and the passive gear 65 to rotate about the first axis (A1) in the second rotational direction (R2). At the same time, the first distal pivot axle 328 pulls the first distal link 325 so that the first intermediate pivot axle 327 of the first linkage 320 is moved toward the distal ends 322 of the first oblong grooves 319 of the first proximal phalange 311. During the movement of the first intermediate pivot axle 327 toward the distal ends 322 of the first oblong grooves 319, the first intermediate link 324, the first proximal pivot axle 326, the first proximal link 323 and the first pin 329 of the first linkage 320 are moved forwardly, so that the first pin 329 of the first linkage 320 is moved to abut against the first stop surfaces 577 of the first constraint member 57.

When the poking arm 582 of the poking member 58 pushes the first protruding block 572 of the first constraint member 57 to rotate the first constraint member 57 in the second rotational direction (R2) to a second rotational position (see FIG. 25), the first stop surfaces 577 of the first limiting plates 575 of the first constraint member 57 still block the first pin 329 of the first linkage 320 of the first finger 31, and the first proximal pivot axle 326 of the first linkage 320 is spaced apart from and coaxial with the second pivot axle 516 (see FIG. 7) of the hinge 51 of the first transmission mechanism 50 along the second axis (A2). At this time, since the first intermediate pivot axle 327 of the first linkage 320 is limited within the distal ends 322 of the first oblong grooves 319 of the first proximal phalange 311, and since the arc-shaped surface 519 of the plate body 515 of the hinge 51 is blocked by the first limiting member 271 of the limiting assembly 27, the first finger 31 is stretched and steadily maintained at the first open position where the first distal end portion 314 of the first finger 31 is distal from the second finger 34.

Referring to FIGS. 25, 26 and 27, during the rotation of the second constraint member 74 in the first rotational direction (R1) from the second pivoting position, the second stop surfaces 747 of the claw portions 746 of the second limiting plates 745 are separated from the second pin 359 of the second finger 34, and outer surfaces of the second limiting plates 745 of the second constraint member 74 are in movable contact with the second pin 359 of the second finger 34. At this time, the second finger 34 is maintained at the second open position. At the same time, the poking arm 743 of the second constraint member 74 pushes the first projection 812 of the release member 81 so as to rotate the release member 81 in the first rotational direction (R1). During the rotation of the re lease member 81 in the first rotational direction (R1), the second projections 813 of the release member 81 respectively push the rollers 843 of the locking modules 84 to the unlocking position, so that the second projections 813 of the release member 81 respectively push the second protrusion 833 and the third protrusion 834 of the inner ring 83 via the rollers 843 to rotate the inner ring 83 in the first rotational direction (R1).

Rotation of the inner ring 83 in the first rotational direction (R1) drives each of the third constraint members 77 to rotate in the first rotational direction (R1) from the first rotating position via the third transmission shaft 75. During the rotation of each of the third constraint members 77 in the first rotational direction (R1) from the first rotating position, the third stop surfaces 776 of each of the third constraint members 77 are rotated to permit movement of the third pin 389 of the corresponding third finger 37. For the sake of brevity, the movement of only one of the third fingers 37 and the corresponding third bias assembly 76 is described in detail. The restoring force generated by the third spring 761 of the third bias assembly 76 moves the third distal pivot axle 388 of the third finger 37 upwardly and rearwardly, so that the third distal phalange 373 is rotated about the pivoting axle 382 relative to the third intermediate phalange 372 in the first rotational direction (R1). At the same time, the third distal pivot axle 388 pulls the third distal link 385 so that the third intermediate pivot axle 387 of the third linkage 380 moves toward the distal ends 378′ of the third oblong grooves 376′ of the third intermediate phalange 372, and that the third proximal pivot axle 386 of the third linkage 380 moves toward the distal ends 378 of the third oblong grooves 376 of the third proximal phalange 371. During the movement of the third intermediate pivot axle 387toward the distal ends 378′ of the third oblong grooves 376′, the third intermediate phalange 372 is rotated about the pivoting axle 379 relative to the third proximal phalange 371 in the first rotational direction (R1). During the movement of the third proximal pivot axle 386 toward the distal ends 378 of the third oblong grooves 376, the third proximal phalange 371 is rotated about the third axis (A3) in the first rotational direction (R1), and the third pin 389 of the third linkage 380 is moved to abut against the third stop, surfaces 776 of the third constraint member 77.

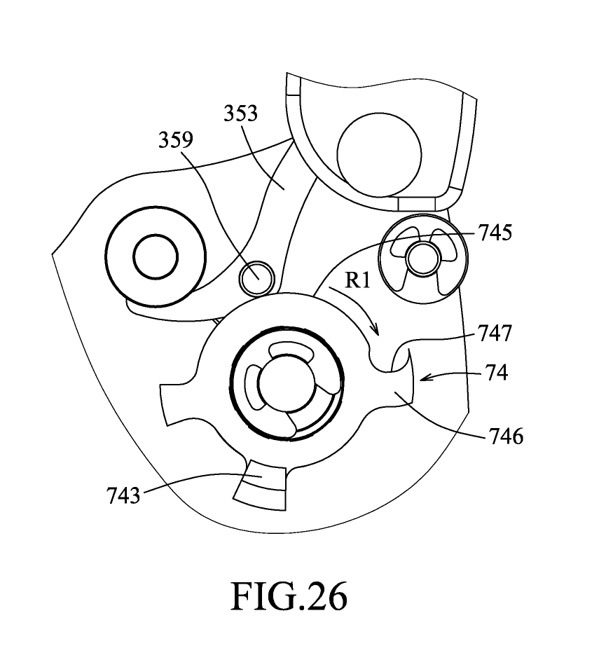

When the first constraint member 57 is rotated in the second rotational direction (R2) to the second rotational position (see FIG. 25), the second constraint member 74 is rotated in the first rotational direction (R1) to a third pivoting position (see FIGS. 25 and 26), the inner ring 83 is rotated in the first rotational direction (R1) to a second angular position (see FIG. 25) by the second constraint member 74 via the release member 81, and each of the third constraint members 77 is rotated in the first rotational direction (R1) to a second rotating position (see FIG. 27) by the inner ring 83 via the third transmission shaft 75. At this time, the third stop surfaces 776 of each of the third constraint members 77 still block the third pin 389 of the corresponding third finger 37. For each of the third fingers 37, since the third intermediate pivot axle 387 of the third linkage 380 is limited within the distal ends 378′ of the third oblong grooves 376′ of the third intermediate phalange 372, since the third proximal pivot axle 386 of the third linkage 380 is limited within the distal ends 378 of the third oblong grooves 376 of the third proximal phalange 371, and since the third proximal end portion 374 of the third finger 37 is blocked by the corresponding third limiting member 274 of the limiting assembly 27, the third finger 37 is stretched and steadily maintained at the third open position.

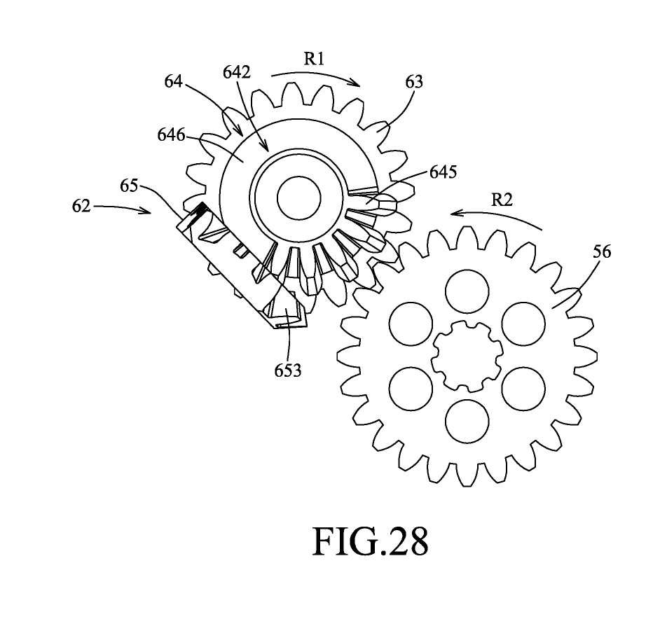

Referring to FIGS. 25 and 28, during the movement of the first constraint member 57 form the first rotational position toward the second rotational position, the intermittent gear set 62 is maintained in the non-meshing state (see FIG. 21) in which the toothed sector 645 of the active gear 64 is separated from the annular gear section 653 of the passive gear 65. Therefore, the passive gear 65 is not rotated when the first constraint member 57 is rotated form the first rotational position toward the second rotational position. When the first constraint member 57 is moved to the second rotational position from the first rotational position, the intermittent gear set 62 is switched into the meshing state (see FIG. 28) in which the toothed sector 645 of the active gear 64 meshes the annular gear section 653 of the passive gear 65.

Referring to FIGS. 20 and 22, rotation of the first transmission gear 56 in the second rotational direction (R2) continuously rotates the second swing arm 95 to move the link assembly 96 downwardly in the push direction (D1) so as to further rotate the first swing arm 94 and the movable gear 93 of the third transmission mechanism 90 about the axial post 23 in the second rotational direction (R2). By such, when the hold unit 3 of the prosthetic hand 100 is switched from the pointing state to the open state, the main frame 2 is continuously rotated relative to the wrist frame 1 in the first swivel direction (S1) by virtue of the meshing between the movable gear 93 and the fixed gear 91.

According to the above, when the hold unit 3 is switched from the pointing state (see FIG. 23) to the open state (see FIG. 25), the first finger 31 is moved from the first closed position to the first open position, and each of the third fingers 37 is moved from the third closed position to the third open position. The second finger 34 is maintained at the second open position. By such, the user can use the first finger 31, the second finger 34 and the third fingers 37 to hold an object.

The following paragraphs describe the switch of the hold unit 3 from the open state (see FIG. 25) to the eversion state (see FIG. 30) where the first finger 31 is at the eversion position, where the second finger 34 is at the second open position, and where each of the third fingers 37 is at the third open position.

Referring to FIGS. 29, 30, 31 and 32, the user continues to contract the muscle of the upper limb, and the first sensor controls the output shaft 411 of the drive motor 41 (see FIG. 6) to rotate in the first rotational direction (R1) upon receipt of the electromyography signal, so as to drive rotation of the first transmission gear 56 of the first transmission mechanism 50 in the second rotational direction (R2), and to drive rotation of the second transmission gear 72 of the second transmission mechanism 70 in the first rotational direction (R1).

The poking arm 582 of the poking member 58 pushes the first protruding block 572 of the first constraint member 57 to rotate the first constraint member 57 in the second rotational direction (R2) from the second rotational position. During the rotation of the first constraint member 57 in the second rotational direction (R2), the first stop surfaces 577 of the first limiting plates 575 of the first constraint member 57 rotate in the second rotational direction (R2) to be separated from the first pin 329 of the first linkage 320 of the first finger 31, and the guide grooves 576 of the first limiting plates 575 of the first constraint member 57 rotate relative to the first pin 329 of the first linkage 320. By virtue of the guide grooves 576 of the first limiting plates 575 of the first constraint member 57, during the rotation of the first constraint member 57 in the second rotational direction (R2) from the second rotational position, the first pin 329 of the first linkage 320 is not moved, and is constrained by the first constraint member 57. At the same time, the intermittent gear set 62 is in the meshing state, and the active gear 64 is rotated by the connecting gear 63 in the first rotational direction (R1) to drive rotation of the passive gear 65 about the second axis (A2) in an eversion direction (T1). Since the truncated surface 652 of the passive gear 65 abuts against the proximal surface 331 of the first proximal end portion 313 of the first finger 31, rotation of the passive gear 65 in the eversion direction (T1) drives synchronous rotation of the first finger body 310 in the eversion direction (T1). In addition, since the first proximal pivot axle 326 of the first linkage 320 is coaxial with the second pivot axle 516 of the hinge 51 of the first transmission mechanism 50, the rotation of the first finger body 310 in the eversion direction (T1) drives the first intermediate link 324 to rotate about the first proximal pivot axle 326 (i.e., about the second axis (A2)) relative to the first proximal link 323.

When the first constraint member 57 is rotated to a third rotational position as shown in FIG. 30, the second side surface 518 of the plate body 515 of the hinge 51 is blocked by the first limiting member 271 so as to prevent further rotation of the first finger body 310. By such, the first finger 31 is still stretched and is steadily maintained at the eversion position where the first distal end portion 314 of the first finger body 310 is distal from the outer surface 211 of the first side plate 21 of the main frame 2.

Referring to FIGS. 30, 33 and 34, on the other hand, during the rotation of the second constraint member 74 in the first rotational direction (R1) from the third pivoting position, the second stop surfaces 747 of the claw portions 746 of the second limiting plates 745 are separated from the second pin 359 of the second finger 34, and outer surfaces of the second limiting plates 745 of the second constraint member 74 are in movable contact with the second pin 359 of the second finger 34. At this time, the second finger 34 is maintained at the second open position. At the same time, the poking arm 743 of the second constraint member 74 pushes the first projection 812 of the release member 81 so as to rotate the release member 81 in the first rotational direction (R1). During the rotation of the release member 81 in the first rotational direction (R1), the second projections 813 of the release member 81 respectively push the rollers 843 of the locking modules 84 to the unlocking position, so that the second projections 813 of the release member 81 respectively push the second protrusion 833 and the third protrusion 834 of the inner ring 83 via the rollers 843 to rotate the inner ring 83 in the first rotational direction (R1).

Rotation of the inner ring 83 in the first rotational direction (R1) drives each of the third constraint members 77 to rotate in the first rotational direction (R1) from the second rotating position via the third transmission shaft 75. During the rotation of each of the third constraint members 77 in the first rotational direction (R1) from the second rotating position, the third stop surfaces 776 of each of the third constraint members 77 are separated from the third pin 389 of the corresponding third finger 37, and the guide grooves 775 of each of the third constraint members 77 are rotated relative the third pin 389 of the corresponding third finger 37. By virtue of the guide grooves 775 of each of the third constraint members 77, during the rotation of each of the third constraint members 77 in the first rotational direction (R1) from the second rotating position, the third pin 389 of the third linkage 380 of the corresponding third finger 37 is not moved, and is constrained by the third constraint members 77. At this time, each of the third fingers 37 is maintained at the third open position.

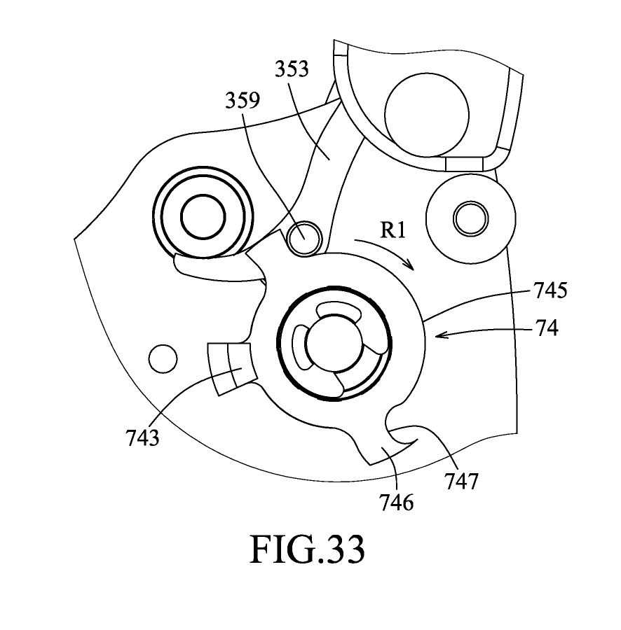

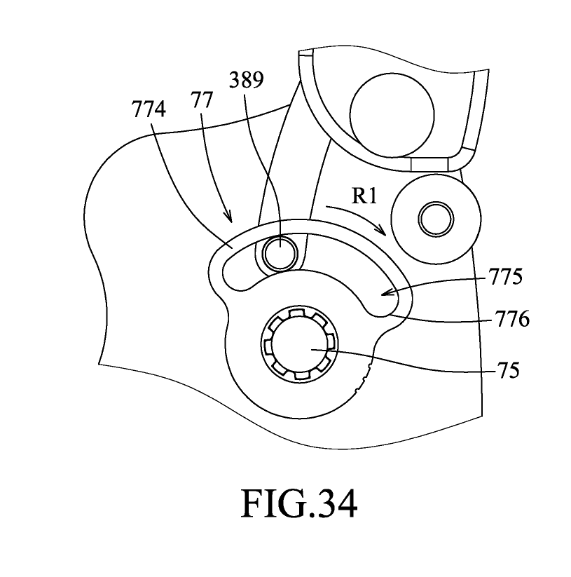

When the first constraint member 57 is rotated in the second rotational direction (R2) to the third rotational position (see FIG. 30), the second constraint member 74 is rotated in the first rotational direction (R1) to a fourth pivoting position (see FIG. 33), the inner ring 83 is rotated in the first rotational direction (R1) to a third angular position (see FIG. 30) by the second constraint member 74 via the release member 81, and each of the third constraint members 77 is rotated in the first rotational direction (R1) to a third rotating position (see FIG. 34) by the inner ring 83 via the third transmission shaft 75.

Referring to FIGS. 29 and 35, rotation of the first transmission gear 56 in the second rotational direction (R2) continuously rotates the second swing arm 95 to move the link assembly 96 downwardly in the push direction (D1) so as to further rotate the first swing arm 94 and the movable gear 93 of the third transmission mechanism 90 about the axial post 23 (see FIGS. 1 and 2) in the second rotational direction (R2). By such, when the hold unit 3 of the prosthetic hand 100 is switched into the eversion state, the link assembly 96 is at the second move position, and the main frame 2 is rotated relative to the wrist frame 1 in the first swivel direction (S1) to the second twist position (see FIG. 35) by virtue of the meshing between the movable gear 93 and the fixed gear 91.

According to the above, when the hold unit 3 is switched from the open state (see FIG. 25) to the eversion state (see FIG. 30), the first finger 31 is moved from the first open position to the eversion position. Each of the third fingers 37 is maintained at the third open position. The second finger 34 is maintained at the second open position. By such, the user can use the first finger 31, the second finger 34 and the third fingers 37 to support a tray, for example.

The following paragraphs describe the switch of the hold unit 3 from the eversion state (see FIG. 30) to the open state (see FIG. 25).

Referring to FIG. 29, the user contracts the muscle of the upper limb, and the second sensor controls the output shaft 411 of the drive motor 41 to rotate in the second rotational direction (R2) upon receipt of the electromyography signal, so as to drive rotation of the first transmission gear 56 of the first transmission mechanism 50 in the first rotational direction (R1), and to drive rotation of the second transmission gear 72 of the second transmission mechanism 70 in the second rotational direction (R2).

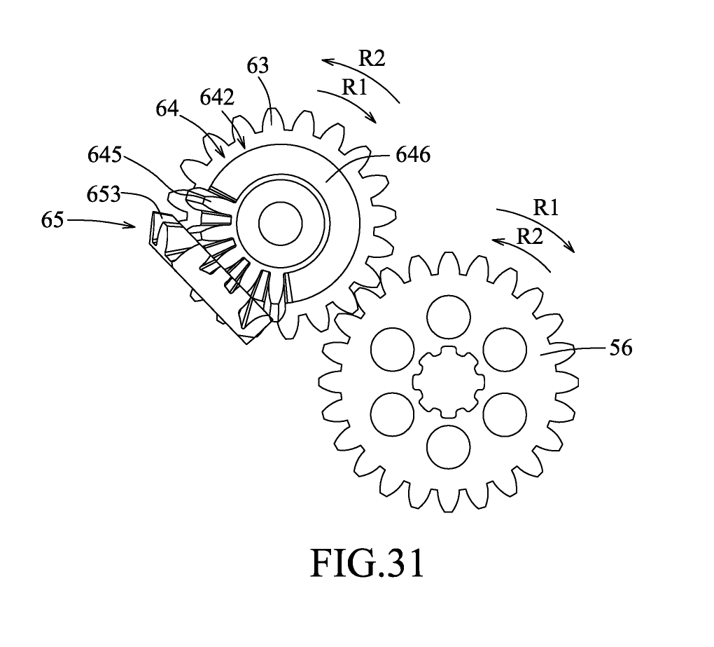

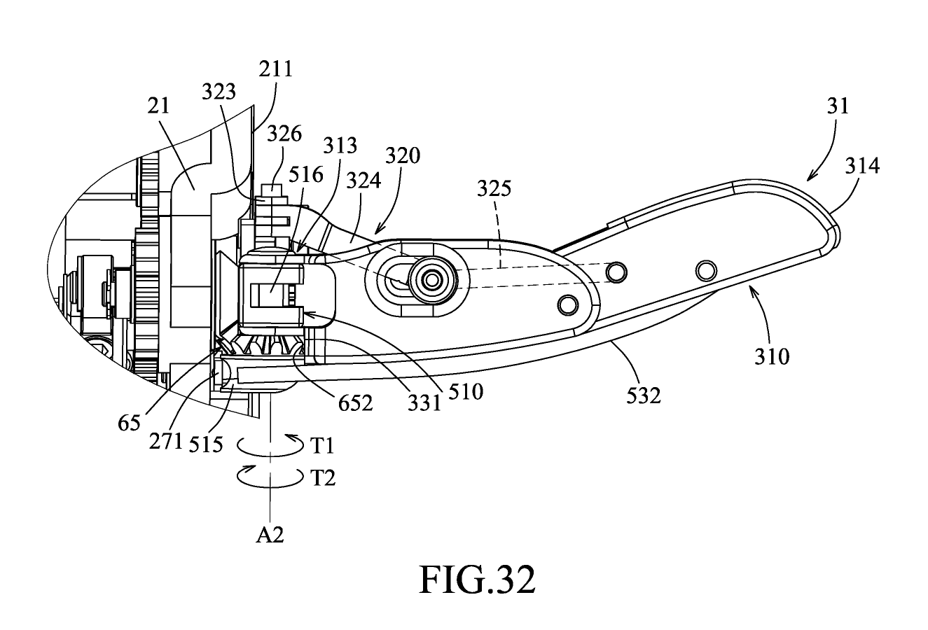

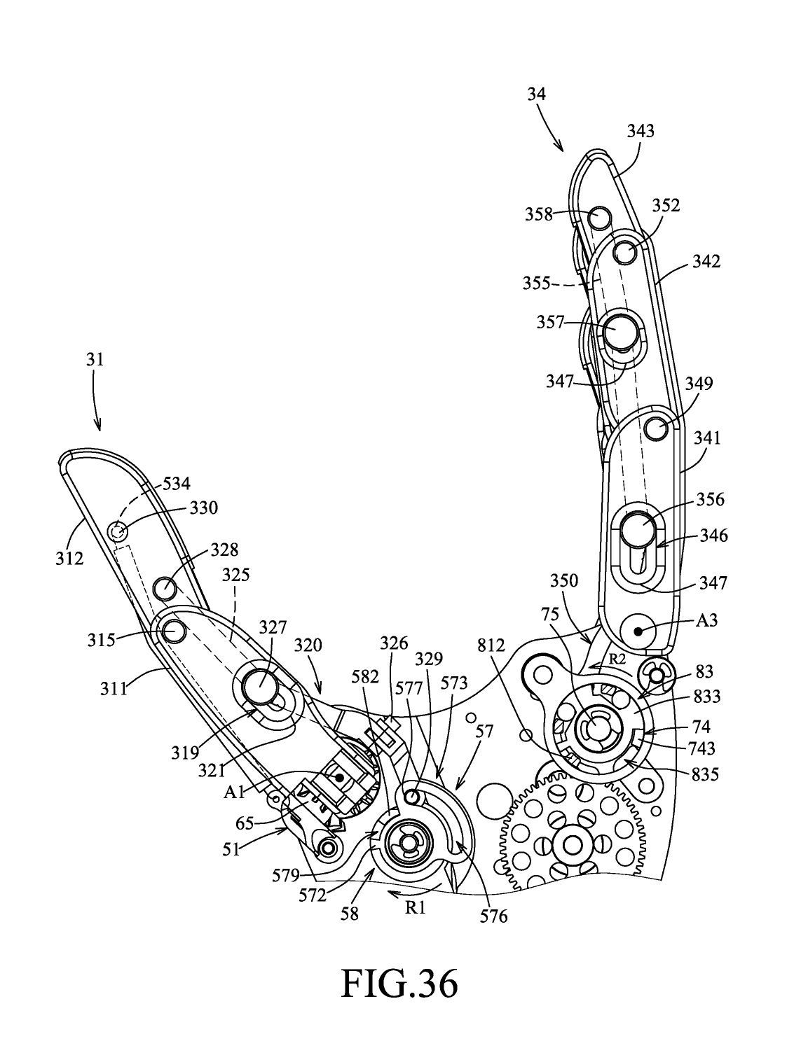

Referring to FIGS. 31, 32 and 36, the rotation of the first transmission gear 56 of the first transmission mechanism 50 in the first rotational direction (R1) drives rotation of the connecting gear 63 in the second rotational direction (R2). The intermittent gear set 62 is in the meshing state, and the active gear 64 is rotated by the connecting gear 63 in the second rotational direction (R2) to drive rotation of the passive gear 65 about the second axis (A2) in an inward direction (T2, see FIG. 32) opposite to the eversion direction (T1). By such, the first finger body 310 is driven to rotate about the second axis (A2) in the inward direction (T2), and the first intermediate link 324 is driven to rotate about the first proximal pivot axle 326 (i.e., about the second axis (A2)) in the inward direction (T2) relative to the first proximal link 323. When the active gear 64 of the intermittent gear set 62 is rotated to the position shown in FIG. 28, the first finger 31 is moved back to the first open position (see FIG. 36). When the first constraint member 57 is moved from the third rotational position to the second rotational position, the intermittent gear set 62 is switched into the non-meshing state.

Referring to FIGS. 30 and 36, since the poking arm 582 of the poking member 58 is spaced apart from the first constraint block 573 of the first constraint member 57 when the hold unit 3 is in the eversion state and when the first finger 31 is at the eversion position, during the rotation of the poking member 58 in the first rotational direction (R1), the poking arm 582 of the poking member 58 is first separated from the first protruding block 572 of the first constraint member 57 and moves within the arc-shaped groove 579 of the first constraint member 57 without rotating the first constraint member 57 until the poking arm 582 of the poking member 58 is in contact with the first constraint block 573, and then pushes the first constraint block 573 to rotate the first constraint member 57 in the first rotational direction (R1) from the third rotational position. By virtue of the guide grooves 576 of the first limiting plates 575 of the first constraint member 57, during the rotation of the first constraint member 57 in the first rotational direction (R1) from the third rotational position, the first pin 329 of the first linkage 320 is not moved until the first constraint member 57 is rotated back to the second rotational position (see FIG. 36) where the first stop surfaces 577 of the first constraint member 57 are in contact with the first pin 329 of the first linkage 320. When the first constraint member 57 is rotated in the first rotational direction (R1) to the second rotational position, the first finger 31 is moved back to the first open position.

Referring to FIGS. 30, 33 and 34, on the other hand, when the second constraint member 74 is at the fourth pivoting position and when the inner ring 83 is at the third angular position, the poking arm 743 of the second constraint member 74 is spaced apart from the second protrusion 833 of the inner ring 83. Therefore, during the rotation of the second constraint member 74 in the second rotational direction (R2) from the fourth pivoting position, the poking arm 743 of the second constraint member 74 is first separated from the first projection 812 of the release member 81 and moves within the arc-shaped retaining groove 835 of the inner ring 83 without rotating the inner ring 83 until the poking arm 743 of the second constraint member 74 is in contact with the second protrusion 833 of the inner ring 83, and then pushes the inner ring 83 to rotate in the second rotational direction (R2) from the third angular position. Rotation of the inner ring 83 in the second rotational direction (R2) drives rotation of each of the third constraint members 77 in the second rotational direction (R2) via the third transmission shaft 75. Since the second stop surfaces 747 of the second limiting plates 745 of the second constraint member 74 are spaced apart from the second pin 359 of the second linkage 350 of the second finger 34, and since the outer surfaces of the second limiting plates 745 of the second constraint member 74 are in movable contact with the second pin 359 of the second finger 34, the rotation of the second constraint member 74 in the second rotational direction (R2) from the fourth pivoting position does not drive movement of the second pin 359 of the second finger 34. By virtue of the guide grooves 775 of the third limiting plates 774 of each of the third constraint members 77, during the rotation of each of the third constraint members 77 in the second rotational direction (R2) from the third rotating position, the third pin 389 of the third linkage 380 of the corresponding third finger 37 is not moved until the third constraint member 77 is rotated back to the second rotating position (see FIG. 27) where the third stop surfaces 776 of the third constraint member 77 are in contact with the third pin 389 of the third linkage 380 of the corresponding third finger 37.

Referring to FIGS. 24, 27 and 36, when the first constraint member 57 is rotated back to the second rotational position (see FIG. 36), the second constraint member 74 is at the second pivoting position (see FIG. 24), the inner ring 83 is at the second angular position (see FIG. 36), and each of the third constraint members 77 is at the second rotating position (see FIG. 27).

Referring to FIGS. 33, 34 and 36, as mentioned above, since the second stop surfaces 747 of the second constraint member 74 are spaced apart from the second pin 359 of the second finger 34, and since the outer surfaces of the second limiting plates 745 of the second constraint member 74 are in movable contact with the second pin 359 of the second finger 34, the rotation of the second constraint member 74 in the second rotational direction (R2) from the fourth pivoting position to the second pivoting position does not drive movement of the second pin 359 of the second finger 34. By virtue of the guide grooves 775 of the third limiting plates 774 of each of the third constraint members 77, during the rotation of each of the third constraint members 77 in the second rotational direction (R2) from the third rotating position to the second rotating position, the third pin 389 of the third linkage 380 of the corresponding third finger 37 is not moved. In addition, by virtue of the arc-shaped retaining groove 835 of the inner ring 83, the rotation of the second constraint member 74 in the second rotational direction (R2) from the fourth pivoting position to the second pivoting position does not immediately drive rotation of the inner ring 83, such that when the second stop surfaces 747 of the claw portions 746 of the second limiting plates 745 are in contact with the second pin 359 of the second finger 34 upon rotation of the second constraint member 74 in the second rotational direction (R2), the third stop surfaces 776 of each of the third constraint members 77 are simultaneously in contact with the third pin 389 of the third linkage 380 of the corresponding third finger 37. By such, the second finger 34 and the third fingers 37 can be simultaneously and respectively moved back to the second closed position and the third closed position.

Referring to FIGS. 29 and 35, rotation of the first transmission gear 56 in the first rotational direction (R1) rotates the second swing arm 95 to move the link assembly 96 upwardly in a pull direction (D2) opposite to the push direction (D1) so as to rotate the first swing arm 94 and the movable gear 93 of the third transmission mechanism 90 about the axial post 23 in the first rotational direction (R1). By such, when the hold unit 3 of the prosthetic hand 100 is switched from the eversion state to the open state, the main frame 2 is rotated relative to the wrist frame 1 in a second swivel direction (S2) opposite to the first swivel direction (S1) by virtue of the meshing between the movable gear 93 and the fixed gear 91.

According to the above, when the hold unit 3 is switched from the eversion state (see FIG. 30) to the open state (see FIG. 25), the first finger 31 is moved from the eversion position to the first open position. Each of the third fingers 37 is maintained at the third open position. The second finger 34 is maintained at the second open position.

The following paragraphs describe the switch of the hold unit 3 from the open state (see FIG. 25) to the closed state (see FIG. 16).

Referring to FIGS. 16, 29 and 36, the user continues to contract the muscle of the upper limb, and the second sensor controls the output shaft 411 of the drive motor 41 to rotate in the second rotational direction (R2) upon receipt of the electromyography signal, so as to drive rotation of the first transmission gear 56 of the first transmission mechanism 50 in the first rotational direction (R1) to further rotate the first constraint member 57 in the first rotational direction (R1), and to drive rotation of the second transmission gear 72 of the second transmission mechanism 70 in the second rotational direction (R2) to further rotate the second constraint member 74 in the second rotational direction (R2).

During the rotation of the first constraint member 57 in the first rotational direction (R1) from the second rotational position, the first stop surfaces 577 of the first constraint member 57 push and move the first pin 329 of the first linkage 320 rearwardly and downwardly, so that the first linkage 320 moves rearwardly and downwardly and that the first intermediate pivot axle 327 of the first linkage 320 moves toward the proximal ends 321 of the first oblong grooves 319 of the first proximal phalange 311. During the movement of the first intermediate pivot axle 327 toward the proximal ends 321 of the first oblong grooves 319, the first proximal phalange 311, and hinge 51 and the passive gear 65 to rotate about the first axis (A1) in the first rotational direction (R1). At the same time, the first intermediate pivot axle 327 of the first linkage 320 pulls the first distal pivot axle 328 via the first distal link 325, so that the first distal phalange 312 is rotated about the pivoting axle 315 relative to the first proximal phalange 311 in the first rotational direction (R1). The abovementioned movement of the first finger 31 deforms the first spring 531 of the first bias assembly 53, so that the first spring 531 generates the restoring force.

When the first constraint member 57 rotates back to the first rotational position (see FIG. 16), the first intermediate pivot axle 327 of the first linkage 320 is limited within the proximal ends 321 of the first oblong grooves 319 of the first proximal phalange 311, and the first stop surfaces 577 of the first constraint member 57 block the first pin 329 of the first linkage 320 to maintain the first finger 31 at the first closed position.

Referring to FIGS. 16, 24 and 36, on the other hand, during the rotation of the second constraint member 74 in the second rotational direction (R2) from the second pivoting position, the second stop surfaces 747 of the second limiting plates 745 of the second constraint member 74 push and move the second pin 359 of the second linkage 350 of the second finger 34 downwardly and forwardly. By such, the second proximal pivot axle 356 of the second linkage 350 is moved toward the proximal ends 347 of the second oblong grooves 346 of the second proximal phalange 341, and the second intermediate pivot axle 357 of the second linkage 350 is moved toward the proximal ends 347′ of the second oblong grooves 346′ of the second intermediate phalange 342. During the movement of the second proximal pivot axle 356toward the proximal ends 347 of the second oblong grooves 346, the second proximal phalange 341 is rotated about the third axis (A3) in the second rotational direction (R2). During the movement of the second intermediate pivot axle 357 toward the proximal ends 347′ of the second oblong grooves 346′, the second intermediate phalange 342 is rotated about the pivoting axle 349 in the second rotational direction (R2) relative to the second proximal phalange 341. At the same time, the second intermediate pivot axle 357 pulls the second distal pivot axle 358 via the second distal link 355, so that the second distal phalange 343 is rotated about the pivoting axle 352 in the second rotational direction (R2) relative to the second intermediate phalange 342. The abovementioned movement of the second finger 34 deforms the second spring 731 of the second bias assembly 73, so that the second spring 731 generates the restoring force.

When the second constraint member 74 rotates back the first pivoting position (see FIGS. 16 and 17), the second proximal pivot axle 356 of the second linkage 350 is limited within the proximal ends 347 of the second oblong grooves 346 of the second proximal phalange 341, the second intermediate pivot axle 357 of the second linkage 350 is limited within the proximal ends 347′ of the second oblong grooves 346′ of the second intermediate phalange 342, and the second stop surfaces 747 of the second constraint member 74 block the second pin 359 of the second linkage 350 to maintain the second finger 34 at the second closed position.

Referring to FIGS. 27 and 36, during the rotation of the second constraint member 74 in the second rotational direction (R2) from the second pivoting position, the inner ring 83 is rotated in the second rotational direction (R2) from the second angular position to rotate the third constraint members 77 in the second rotational direction (R2) from the second rotating position via the third transmission shaft 75. For the sake of brevity, the movement of only one of the third constraint members 77 and the corresponding third finger 37 is described in detail. During the rotation of the third constraint member 77 in the second rotational direction (R2) from the second rotating position, the third stop surfaces 776 of the third constraint member 77 push and move the third pin 389 of the corresponding third finger 37 downwardly and forwardly. By such, the third proximal pivot axle 386 of the third linkage 380 is moved toward the proximal ends 377 of the third oblong grooves 376 of the third proximal phalange 371, and the third intermediate pivot axle 387 of the third linkage 380 is moved toward the proximal ends 377′ of the third oblong grooves 376′ of the third intermediate phalange 372. During the movement of third proximal pivot axle 386 toward the proximal ends 377 of the third oblong grooves 376, the third proximal phalange 371 is rotated about the third axis (A3) in the second rotational direction (R2). During the movement of the third intermediate pivot axle 387 toward the proximal ends 377′ of the third oblong grooves 376′, the third intermediate phalange 372 is rotated about the pivoting axle 379 in the second rotational direction (R2) relative to the third proximal phalange 371. At the same time, the third intermediate pivot axle 387 pulls the third distal pivot axle 388 via the third distal link 335, so that the third distal phalange 373 is rotated about the pivoting axle 382 in the second rotational direction (R2) relative to the third intermediate phalange 372. The abovementioned movement of the third finger 37 deforms the third spring 761 of the third bias assembly 76, so that the third spring 761 generates the restoring force.

When the inner ring 83 is rotated back to the first angular position (see FIG. 16), the third constraint member 77 rotates back the first rotating position (see FIG. 18). At this time, the third proximal pivot axle 386 of the third linkage 380 is limited within the proximal ends 377 of the third oblong grooves 376 of the third proximal phalange 371, the third intermediate pivot axle 387 of the third linkage 380 is limited within the proximal ends 377′ of the third oblong grooves 346′ of the third intermediate phalange 372, and the third stop surfaces 776 of the third constraint member 77 block the third pin 389 of the corresponding third finger 37 to maintain the corresponding third finger 37 at the third closed position.

Referring to FIGS. 19 and 20, when the first constraint member 57 rotates back to the first rotational position (see FIG. 16), the link assembly 96 is moved back to the first move position (see FIG. 19), and the main frame 2 is rotated relative to the wrist frame 1 back to the first twist position (see FIG. 20).

According to the above, when the hold unit 3 is switched from the open state (see FIG. 25) to the closed state (see FIG. 16), the first finger 31 is moved back to the first closed position, the second finger 34 is moved back to the second closed position, each of the third fingers 37 is moved back to the third closed position, and the main frame 2 is rotated relative to the wrist frame 1 back to the first twist position (see FIG. 20).

By virtue of the operation of the first sensor and the second sensor, the user can switch the hold unit 3 of the prosthetic hand 100 between any two of the states illustrated above. Some examples are as follows:

To switch the hold unit 3 between the closed state and the pointing state, the user could initiate operation of the first sensor to control the drive unit 4 to move the second finger 34 from the second closed position to the second open position, and then stop the operation of the first sensor so as to maintain the second finger 34 at the second open position. After use of the second finger 34, the user could initiate operation of the second sensor to control the drive unit 4 so as to move the second finger 34 from the second open position to the second closed position. By such, the second finger 34 is movable between the second closed position and the second open position while the first finger 31 and the third fingers 37 are maintained at the first closed position and the third closed position, respectively.

To switch the hold unit 3 between the open state and the eversion state, the user could initiate operation of the first sensor to control the drive unit 4 to move the first finger 31 from the first open position to the eversion position, and then stop the operation of the first sensor so as to maintain the first finger 31 at the eversion position. Afterwards, the user could initiate operation of the second sensor to control the drive unit 4 so as to move the first finger 31 from the eversion position to the first open position. By such, the first finger 31 is movable between the first open position and the eversion position while the second finger 34 and the third fingers 37 are maintained at the second open position and the third open position, respectively.

It should be noted that the first finger 31, the second finger 34 and the third fingers 37 can also be maintained at an arbitrary position via operation of the first sensor and the second sensor. For example, when the first sensor is operated to control the drive unit 4 so as to move the second finger 34 from the second open position toward the second closed position, the second finger 34 can be maintained at any position between the second open position and the second closed position by timely stopping the operation of the first sensor.

In summary, the prosthetic hand 100 of this disclosure is operable to switch among various states for different demands of a user. In addition, the switch of the prosthetic hand 100 among the various states is implemented by a single actuator (i e, the drive motor 41), so the maintenance expense of the prosthetic hand 100 is relatively low.

In the description above, for the purposes of explanation, numerous specific details have been set forth in order to provide a thorough understanding of the embodiment. It will be apparent, however, to one skilled in the art, that one or more other embodiments maybe practiced without some of these specific details. It should also be appreciated that reference throughout this specification “one embodiment,” “an embodiment,” an embodiment with an indication of an ordinal number and so forth means that a particular feature, structure, or characteristic may be included in the practice of the disclosure. It should be further appreciated that in the description, various features are sometimes grouped together in a single embodiment, figure, or description thereof for the purpose of streamlining the disclosure and aiding in the understanding of various inventive aspects.

While the disclosure has been described in connection with what is considered the exemplary embodiment, it is understood that this disclosure is not limited to the disclosed embodiment but is intended to cover various arrangements included within the spirit and scope of the broadest interpretation so as to encompass all such modifications and equivalent arrangements.

What is claimed is:

1. A prosthetic hand comprising:
a main frame having an outer surface;
a hold unit including a first finger, and a second finger that corresponds in position to said first finger, said first finger having a first proximal end portion that is proximate to said outer surface, and a first distal end portion that is opposite to said first proximal end portion, said second finger being disposed on said main frame; and
a drive unit including a drive mechanism and a first transmission mechanism, said drive mechanism being disposed on said main frame, said first transmission mechanism being disposed on said main frame, and being coupled to said drive mechanism and said first finger, said drive mechanism driving said first transmission mechanism to rotate said first finger about a first axis between a first closed position and a first open position, and to rotate said first finger about a second axis that is transverse to the first axis between the first open position and an eversion position, said first distal end portion of said first finger being proximate to said second finger when said first finger is at the first closed position, said first distal end portion of said first finger being distal from said second finger when said first finger is at the first open position, said first distal end portion of said first finger being distal from said outer surface when said first finger is at the eversion position.

2. The prosthetic hand as claimed in claim 1, wherein said second finger is rotatably mounted to said main frame, said drive unit further including a second transmission mechanism that is coupled to said drive mechanism and said second finger, said drive mechanism driving said second transmission mechanism to rotate said second finger about a third axis that is parallel to the first axis between a second closed position and a second open position, said second finger being proximate to said first finger when said second finger is at the second closed position, said second finger being distal from said first finger when said second finger is at the second open position.

3. The prosthetic hand as claimed in claim 2, wherein said hold unit is operable to switch among a closed state, a pointing state, an open state and an eversion state, when said hold unit is in the closed state, said first finger being at the first closed position and said second finger being at the second closed position, when said hold unit is in the pointing state, said first finger being at the first closed position and said second finger being at the second open position, when said hold unit is in the open state, said first finger being at the first open position and said second finger being at the second open position, when said hold unit is in the eversion state, said first finger being at the eversion position and said second finger being at the second open position.

4. The prosthetic hand as claimed in claim 3, wherein said hold unit further includes a third finger that is rotatably mounted to said main frame, said third finger being spaced apart from said second finger along the third axis, and being coupled to said second transmission mechanism, said drive mechanism driving said second transmission mechanism to rotate said third finger about the third axis between a third closed position and a third open position, said third finger being proximate to said first finger when said third finger is at the third closed position, said third finger being distal from said first finger when said third finger is at the third open position, said third finger being at the third closed position when said hold unit is in the closed state or in the pointing state, said third finger is at the third open position when said hold unit is in the open state or in the eversion state.

5. The prosthetic hand as claimed in claim 1, further comprising a wrist frame, said main frame being rotatably mounted to said wrist frame, said drive unit further including a third transmission mechanism, said third transmission mechanism being coupled to said first transmission mechanism, said main frame and said wrist frame, said drive mechanism driving said first transmission mechanism and said third transmission mechanism to rotate said main frame relative to said wrist frame about a fourth axis that is orthogonal to said first axis between a first twist position and a second twist position.

6. The prosthetic hand as claimed in claim 1, wherein said first finger includes a first finger body, and a first linkage that is connected to said first finger body, said first finger body having said first proximal end portion and said first distal end portion, said first proximal end portion being proximate to said outer surface of said main frame, said first transmission mechanism including a hinge, a first bias assembly and a constraint assembly, said hinge being connected to said main frame and said first proximal end portion of said first finger, and defining the first axis, said first bias assembly being coupled to said main frame and said first finger for resiliently biasing said first finger to move toward the first open position, said constraint assembly being coupled to said drive mechanism for constraining said first linkage, said drive mechanism driving said constraint assembly to move said first linkage, so that said first finger being able to be biased by said first bias assembly to move from the first closed position to the first open position, or be driven by said constraint assembly to move from the first open position to the first closed position via said first linkage.

7. The prosthetic hand as claimed in claim 6, wherein said hinge further defines the second axis that is perpendicular to the first axis, said first transmission mechanism further including a turning assembly that is coupled to said constraint assembly and said first proximal end portion of said first finger, said turning assembly being driven by said constraint assembly to move said first finger between the first open position and the eversion position, said first bias assembly including a first spring that resiliently biases said first finger to move toward the first open position.

8. The prosthetic hand as claimed in claim 6, wherein said first linkage includes a first pin that is proximate to said first proximal end portion of said first finger, said main frame including a first side plate that has said outer surface and that further has an inner surface opposite to said outer surface, said hinge being rotatably mounted to said first side plate, said constraint assembly including a first transmission shaft, a first transmission gear, a first constraint member and a poking member, said first transmission shaft being parallel to the first axis and being rotatably mounted to said first side plate, said first transmission gear being co-rotatably mounted to said first transmission shaft and being located at an inner side of said inner surface of said first side plate, said first transmission gear being coupled to and driven by said drive mechanism, said first constraint member being rotatably mounted to said first transmission shaft and being located at an outer side of said outer surface of said first side plate, said first constraint member having at least one first stop surface that serves to block or push said first pin, said poking member being co-rotatably sleeved on said first transmission shaft, said poking member serving to push said first constraint member for driving or permitting movement of said first pin of said first linkage, said first stop surface of said first constraint member blocking said first pin of said first linkage when said first finger is at the first closed position or at the first open position, said first stop surface of said first constraint member being separated from said first pin of said first linkage when said first finger is at the eversion position.

9. The prosthetic hand as claimed in claim 8, wherein said first constraint member has a first protruding block, and a first constraint block that has said first stop surface and that is spaced apart from said first protruding block, said poking member having a poking arm that is located between said first protruding block and said first constraint block of said first constraint member, said poking arm of said poking member serving to push said first protruding block of said first constraint member for rotating said first constraint member from a first rotational position to a second rotational position, or to push said first constraint block of said first constraint member for rotating said first constraint member from the second rotational position to the first rotational position, when said first constraint member is at the first rotational position, said first stop surface of said first constraint member blocking said first pin of said first linkage for maintaining said first finger at the first closed position, when said first constraint member is at the second rotational position, said first stop surface of said first constraint member blocking said first pin of said first linkage for maintaining said first finger at the first open position.

10. The prosthetic hand as claimed in claim 9, wherein said hinge further defines the second axis that is perpendicular to the first axis, said first transmission mechanism further including a turning assembly, said turning assembly including an intermittent gear set and a connecting gear, said intermittent gear set being disposed between said first side plate and said hinge, and being connected to said first proximal end portion of said first finger for intermittently turning said first finger body of said first finger about the second axis, said connecting gear meshing with said first transmission gear and being coupled to said intermittent gear set for driving said intermittent gear set to switch between a non-meshing state in which the rotation of said first transmission gear does not drive movement of said first finger body, and a meshing state in which the rotation of said first transmission gear drives rotation of said first finger body about the second axis, said intermittent gear set being in the non-meshing state when said first finger is at the first closed position, said intermittent gear set being switched from the non-meshing state into the meshing state when said first finger is moved to the first open position from the first closed position, said intermittent gear set being in the meshing state when said first finger is at the eversion position, said intermittent gear set being switched from the meshing state into the non-meshing state when said first finger is moved to the first open position from the eversion position, said poking arm of said poking member further serving to push said first protruding block of said first constraint member for rotating said first constraint member from the second rotational position to a third rotational position, or to push said first constraint block of said first constraint member for rotating said first constraint member from the third rotational position to the second rotational position, when said first constraint member is at the third rotational position, said first stop surface of said first constraint member being separated from said first pin of said first linkage, and said first finger being at the eversion position.

11. The prosthetic hand as claimed in claim 10, wherein said first constraint member further has a first barrel that is rotatably mounted to said first transmission shaft, said first protruding block and said first constraint block being disposed on an outer surrounding surface of said first barrel, and being angularly spaced apart from each other, said first constraint block including two first limiting plates that are spaced apart from each other in the axial direction of said first transmission shaft, said first limiting plates being respectively located at two opposite lateral sides of said first linkage for limiting wobble movement of said first linkage, each of said first limiting plates being formed with an arc-shaped guide groove, said first stop surface being located at an end of said guide groove of one of said first limiting plates, said first pin of said first linkage engaging said guide grooves of said first limiting plates, and being movable along said guide grooves.

12. The prosthetic hand as claimed in claim 11, wherein said first linkage further includes a first proximal link, a first intermediate link, a first proximal pivot axle and a first intermediate pivot axle, said first proximal link being located between said first limiting plates of said first constraint member, said first pin extending through a proximal portion of said first proximal link, a proximal portion of said first intermediate link being pivotally connected to a distal portion of said first proximal link by virtue of said first proximal pivot axle, a distal portion of said first intermediate link being connected to said first finger body by virtue of said first intermediate pivot axle, said first intermediate pivot axle being orthogonal to said first proximal pivot axle, and parallel to the first axis, when said first finger is at the first open position, said first proximal pivot axle being coaxial with the second axis, so that said first intermediate link rotates relative to said first proximal link about said first proximal pivot axle during the movement of said first finger between the first open position and the eversion position.

13. The prosthetic hand as claimed in claim 12, wherein said first finger body includes a first proximal phalange, a first distal phalange and a pin member, said first proximal phalange having said first proximal end portion, and being formed with a first oblong groove, said first distal phalange being pivotally connected to a distal portion of said first proximal phalange, and having said first distal end portion, said pin member being mounted to said first distal phalange and being spaced apart from said first distal end portion, said first intermediate pivot axle engaging said first oblong groove of said first proximal phalange and being movable along said first oblong groove, said first linkage further including a first distal link and a first distal pivot axle, a distal portion of said first intermediate link being pivotally connected to a proximal portion of said first distal link by virtue of said first intermediate pivot axle, a distal portion of said first distal link being pivotally connected to said first distal phalange by virtue of said first distal pivot axle, said first finger being bent when said first finger is at the first closed position, said first finger being stretched when said first finger is at the first open position or at the eversion position, said main frame further including a support arm that is disposed on said outer surface and that is proximate to said first proximal end portion of said first finger, said first bias assembly including a first spring that is configured as an extension spring and that has two opposite end respectively connected to said pin member and said support arm.

14. The prosthetic hand as claimed in claim 10, wherein said hinge includes a first pivoting module and a second pivoting module, said first pivoting module including an axle block, and a first pivot axle that is mounted to said axle block and that defined the first axis, said second pivoting module including a plate body, and a second pivot axle that defines the second axis, said second pivot axle extending through said first proximal end portion of said first finger, said axle block and said plate body, said intermittent gear set including an active gear and a passive gear, said active gear having a hollow axle and a gear body that are coaxially arranged, said hollow axle of said active gear being rotatably mounted to said first side plate and being co-rotatably connected to said connecting gear, said hollow axle and said gear body cooperatively defining a pivoting hole that permits said first pivot axle of said first pivoting module of said hinge to rotatably extend therethrough, said passive gear permitting said second pivot axle to extend therethrough, and being co-rotatably mounted to said first proximal end portion of said first finger, said main frame further including a first limiting member that is for blocking said plate body so as to maintain said first finger at the first open position or at the eversion position.

15. The prosthetic hand as claimed in claim 10, wherein said main frame further includes an axial rod that defines a third axis parallel to the first axis, said second finger being rotatably mounted to said axial rod of said main frame, and being located at the outer side of said outer surface of said first side plate, said drive unit further including a second transmission mechanism that is coupled to said drive mechanism and said second finger, said drive mechanism driving said second transmission mechanism to rotate said second finger about the third axis between a second closed position and a second open position, said second finger being proximate to said first finger when said second finger is at the second closed position, said second finger being distal from said first finger when said second finger is at the second open position.

16. The prosthetic hand as claimed in claim 15, wherein said second finger includes a second finger body that is rotatably mounted to said axial rod of said main frame, and a second linkage that is connected to said second finger body, said second transmission mechanism including a second transmission shaft that is rotatably mounted to said first side plate, a second transmission gear that is co-rotatably mounted to said second transmission shaft, that is located at the inner side of said inner surface of said first side plate, and that is coupled to said drive mechanism, a second bias assembly, and a second constraint member that is co-rotatably mounted to said second transmission shaft, that is located at the outer side of said outer surface of said first side plate, and that is for constraining said second linkage of said second finger, said second bias assembly being coupled to said main frame and said second finger for resiliently biasing said second finger to move toward the second open position, said second transmission shaft rotating said second constraint member for driving said second linkage, so that said second finger being able to be biased by said second bias assembly to move from the second closed position to the second open position, or to be driven by said second constraint member to move from the second open position to the second closed position via said second linkage.

17. The prosthetic hand as claimed in claim 16, wherein said second finger body has a second proximal end portion that is located at the outer side of said outer surface of said first side plate, and a second distal end portion that is opposite to said second proximal end portion, said second linkage including a second pin that is proximate to said second distal end portion of said second finger body, said second constraint member having at least one second stop surface that serves to block or push said second pin, said second transmission shaft rotating said second constraint member for driving or permitting movement of said second pin of said second linkage, said second stop surface of said second constraint member blocking said second pin of said second linkage when said second finger is at the second closed position, said second stop surface of said second constraint member being separated from said second pin of said second linkage when said first finger is at the eversion position.

18. The prosthetic hand as claimed in claim 17, wherein said first constraint member further has an arc-shaped groove that is defined between said first protruding block and said first constraint block and that permits said poking arm of said poking member to movably extend thereinto, said second transmission shaft rotating said second constraint member between a first pivoting position and a second pivoting position, when said second constraint member is at the first pivoting position, said second finger being at the second closed position, said first constraint member being at the first rotational position, and said poking arm of said poking member abutting against said first constraint block and being spaced apart from said first protruding block, when said second constraint member is at the second pivoting position, said second finger being at the second open position.

19. The prosthetic hand as claimed in claim 18, wherein said second constraint member has a second barrel that is co-rotatable sleeved on said second transmission shaft, a second constraint block that is disposed on an outer surrounding surface of said second barrel, said second constraint block including two second limiting plates that are spaced apart from each other in the axial direction of said second transmission shaft, said second limiting plates being respectively located at two opposite lateral sides of said second linkage for limiting wobble movement of said second linkage, each of said second limiting plates having said second stop surface.

20. The prosthetic hand as claimed in claim 19, wherein said second finger body including a second proximal phalange, a second intermediate phalange and a second distal phalange, said second linkage including a second proximal link, a second intermediate link, a second distal link, a second proximal pivot axle, a second intermediate pivot axle and a second distal pivot axle, said second proximal phalange having said second proximal end portion and being rotatably mounted to said axial rod of said main frame, a proximal portion of said second intermediate phalange being pivotally connected to a distal portion of said second proximal phalange, said second distal phalange having said second distal end portion, a proximal portion of said second distal phalange being pivotally connected to a distal portion of said second intermediate phalange, said second proximal phalange being formed with a second oblong groove, said second intermediate phalange being formed with a second oblong groove, said second proximal pivot axle engaging said second oblong groove of said second proximal phalange, and being movable along said second oblong groove of said second proximal phalange, said second intermediate pivot axle engaging said second oblong grooves of said second intermediate phalange, and being movable along said second oblong grooves of said second intermediate phalange, a proximal portion of said second intermediate link being pivotally connected to a distal portion of said second proximal link by virtue of said second proximal pivot axle, a proximal portion of said second distal link being pivotally connected to a distal portion of said second intermediate link by virtue of said second intermediate pivot axle, a distal portion of said second distal link being pivotally connected to said second distal phalange by virtue of said second distal pivot axle, said second proximal link being located between said second limiting plates of said second constraint member, said second pin extending through a proximal portion of said second proximal link, said second finger being bent when said second finger is at the second closed position, said second finger being stretched when said second finger is at the second open position, said main frame further including an insertion pin that is mounted to said first sideplate and that is proximate to said axial rod, said second bias assembly including a second spring that is configured as an extension spring and that has two opposite end respectively connected to said second distal pivot axle and said insertion pin.

21. The prosthetic hand as claimed in claim 16, wherein said main frame further includes a second 1 limiting member, said second limiting member being for blocking said second proximal end portion of said second finger so as to maintain said second finger at the second open position.

22. The prosthetic hand as claimed in claim 19, wherein said hold unit further includes a third finger that is located at the inner side of said inner surface of said first side plate and that is rotatably mounted to said axial rod of said main frame, said drive mechanism driving said second transmission mechanism to rotate said third finger about the third axis between a third closed position and a third open position, said third finger being proximate to said first finger when said third finger is at the third closed position, said third finger being distal from said first finger when said third finger is at the third open position, said third finger including a third finger body that is rotatably mounted to said axial rod of said main frame, and a third linkage that is connected to said third finger body, said second transmission shaft being formed with a communication hole, said second transmission mechanism further including an additory transmission shaft that rotatably extends through said communication hole of said second transmission shaft, an additory bias assembly, an additory constraint member that is co-rotatably mounted to said additory transmission shaft and that is located at the inner side of said first side plate, and a transmission assembly that is coupled to said additory transmission shaft and that is located at the outer side of said second constraint member, said additory bias assembly being coupled to said main frame and said third finger for resiliently biasing said third finger to move toward the third open position, said second constraint member actuating said additory transmission shaft via said transmission assembly so as to rotate said additory constraint member for driving said third linkage of said third finger, so that said third finger being able to be biased by said additory bias assembly to move from the third closed position to the third open position, or to be driven by said additory constraint member to move from the third open position to the third closed position via said third linkage.

23. The prosthetic hand as claimed in claim. 22, wherein said third finger body has a third proximal end portion that is located at the inner side of said first side plate, and a third distal end portion that is opposite to said third proximal end portion, said third linkage includes a third pin that is proximate to said third proximal end portion, said additory constraint member having at least one additory stop surface that serves to block or push said third pin of said third linkage, said additory transmission shaft rotating said additory constraint member for driving or permitting movement of said third pin of said third linkage, said additory stop surface of said additory constraint member blocking said third pin of said third linkage when said third finger is at the third closed position, said additory stop surface of said additory constraint member being separated from said third pin of said third linkage when said first finger is at the eversion position.

24. The prosthetic hand as claimed in claim 23, wherein said additory constraint member has an additory barrel that is co-rotatably sleeved on said additory transmission shaft, and an additory constraint block that is disposed on an outer surrounding surface of said additory barrel, said additory constraint block including two additory limiting plates that are spaced apart from each other in the axial direction of said additory transmission shaft, said additory limiting plates being respectively located at the opposite lateral sides of said third linkage of said third finger for limiting wobble movement of said third linkage, each of said additory limiting plates being formed with an arc-shaped guide groove, said additory stop surface being located at an end of one of said arc-shaped guide grooves of said additory limiting plates, said third pin of said third linkage engaging said arc-shaped guide grooves of said additory limiting plates, and being movable along said arc-shaped guide grooves of said additory limiting plates.

25. The prosthetic hand as claimed in claim 23, wherein said transmission assembly includes a clutch and a release member, when said second constraint member rotates such that said second finger moves from the second closed position to the second open position, said clutch preventing rotation of said additory transmission shaft so as to maintain said third finger at the third closed position, said second constraint member being operable to unlock said clutch via said release member, so as to drive rotation of said additory constraint member via said clutch and said additory transmission shaft for moving said third finger from the third closed position to the third open position.

26. The prosthetic hand as claimed in claim 25, wherein said clutch includes an outer ring that is fixedly mounted to said outer surface of said main frame, an inner ring that is disposed in said outer ring and that is co-rotatably sleeved on said additory transmission shaft, and a plurality of locking modules each of which is disposed between said outer ring and said inner ring, each of said locking modules being operable to switch between a locking state in which said inner ring is permitted to rotate relative to said outer ring in only one direction, and an unlocking state in which said inner ring is freely rotatable relative to said outer ring, said inner ring having a first protrusion and a second protrusion that is spaced apart from said first protrusion, said inner ring further having an arc-shaped retaining groove that is defined between said first protrusion and said second protrusion, said release member including a cover plate that is rotatably sleeved on said additory transmission shaft and that covers said inner ring, a first projection that projects from an inner surface of said cover plate, and a plurality of second projections that project from the inner surface of said cover plate, said first projection of said release member being movably received in said arc-shaped retaining groove of said inner ring, said second constraint member further having a poking arm that is disposed on an outer periphery of said second barrel, that is movably received in said arc-shaped retaining groove of said inner ring, and that is located between said second protrusion of said inner ring and said first projection of said release member, said poking arm of said second constraint member being operable to push said first projection of said release member so that said second projections respectively unlock said locking modules for moving said third finger from the third closed position to the third open position, and to push said second protrusion of said inner ring for moving said third finger from the third open position to the third closed position.

27. The prosthetic hand as claimed in claim 26, wherein:
when said second constraint member is at the first pivoting position, said inner ring is at a first angular position, said first projection of said release member is proximate to said first protrusion of said inner ring, said poking arm of said second constraint member abuts against said second protrusion of said inner ring and is spaced apart from said first projection of said release member, each of said locking modules is in the locking state, said third finger is at the third closed position, and said hold unit is in a closed state;
when said second transmission shaft rotates said second constraint member from the first pivoting position to the second pivoting position in a first rotational direction, said first constraint member is at the first rotational position, said poking arm of said poking member abuts against said first protruding block of said first constraint member and spaced apart from said first constraint block of said first constraint member, said inner ring is at a first angular position, said poking arm of said second constraint member abuts against said first projection of said release member and is spaced apart from said second protrusion of said inner ring, each of said locking modules is in the locking state, and said hold unit is switched into a pointing state; and
when said second transmission shaft rotates said second constraint member from the second pivoting position to the first pivoting position in a second rotational direction opposite to the first rotational direction, said second stop surface of said second constraint member pushes and moves said second pin of said second finger to move said second finger to the second closed position, and said hold unit is switched into the closed state.

28. The prosthetic hand as claimed in claim 27, wherein :
when said second transmission shaft rotates said second constraint member from the second pivoting position in the first rotational direction to a third pivoting position, said poking arm of said poking member pushes said first protruding block of said first constraint member to rotate said first constraint member from the first rotational position to the second rotational position, said second stop surface of said second constraint member is separated from said second pin of said second finger, said poking arm of said second constraint member pushes said first projection of said release member to rotate said release member in the first rotational direction so that said second projections of said release member respectively unlock said locking modules and that said inner ring is moved to a second angular position, said inner ring rotates said additory constraint member via said additory transmission shaft so as to move said third finger to the third open position, and said hold unit is switched into an open state; and
when said second transmission shaft rotates said second constraint member from the third pivoting position in the first rotational direction to a fourth pivoting position, said poking arm of said poking member pushes said first protruding block of said first constraint member to rotate said first constraint member to the third rotational position, said poking arm of said second constraint member pushes said first projection of said release member to rotate said release member in the first rotational direction so that said second projections of said release member respectively push said locking modules to move said inner ring to a third angular position, said inner ring rotates said additory constraint member via said additory transmission shaft so as to separate said additory stop surface from said third pin of said third finger, and said hold unit is switched into an eversion state.

29. The prosthetic hand as claimed in claim 28, wherein:
when said second transmission shaft rotates said second constraint member from the fourth pivoting position in the second rotational direction to the second pivoting position, said poking arm of said poking member pushes said first constraint block of said first constraint member to rotate said first constraint member to the second rotational position, said poking arm of said second constraint member pushes said second protrusion of said inner ring to rotate said inner ring to the second angular position, said inner ring rotates said additory constraint member via said additory transmission shaft so that said additory stop surface is in contact with said third pin of said third finger, and said hold unit is switched into the open state; and
when said second transmission shaft rotates said second constraint member from the second pivoting position in the second rotational direction to the first pivoting position, said poking arm of said poking member pushes said first protruding block of said first constraint member to rotate said first constraint member to the first rotational position, said second stop surface of said second constraint member pushes said second pin of said second finger so as to move said second finger to the second closed position, said poking arm of said second constraint member pushes said second protrusion of said inner ring to rotate said inner ring to the first angular position, said inner ring rotates said additory constraint member via said additory transmission shaft so that said additory stop surface pushes said third pin of said third finger to move said third finger to the third closed position, and said hold unit is switched into the closed state.

30. The prosthetic hand as claimed in claim 24, wherein said third finger body including a third proximal phalange, a third intermediate phalange and a third distal phalange, said third linkage including a third proximal link, a third intermediate link, a third distal link, a third proximal pivot axle, a third intermediate pivot axle and a third distal pivot axle, said third proximal phalange having said third proximal end portion and being rotatably mounted to said axial rod of said mainframe, a proximal portion of said third intermediate phalange being pivotally connected to a distal portion of said third proximal phalange, said third distal phalange having said third distal end portion, a proximal portion of said third distal phalange being pivotally connected to a distal portion of said third intermediate phalange, said third proximal phalange being formed with a third oblong groove, said third intermediate phalange being formed with a third oblong groove, said third proximal pivot axle engaging said third oblong groove of said third proximal phalange, and being movable along said third oblong groove of said third proximal phalange, said third intermediate pivot axle engaging said third oblong grooves of said third intermediate phalange, and being movable along said third oblong grooves of said third intermediate phalange, a proximal portion of said third intermediate link being pivotally connected to a distal portion of said third proximal link by virtue of said third proximal pivot axle, a proximal portion of said third distal link being pivotally connected to a distal portion of said third intermediate link by virtue of said third intermediate pivot axle, a distal portion of said third distal link being pivotally connected to said third distal phalange by virtue of said third distal pivot axle, said third proximal link being located between said additory limiting plates of said additory constraint member, said third pin extending through a proximal portion of said third proximal link, said third finger being bent when said third finger is at the third closed position, said third finger being stretched when said third finger is at the third open position, said main frame further including an insertion pin that is mounted to said first side plate and that is proximate to said axial rod, said additory bias assembly including a additory spring that is configured as an extension spring and that has two opposite end respectively connected to said third distal pivot axle and said insertion pin.

31. The prosthetic hand as claimed in claim 22, wherein said main frame further includes a third limiting member, said third limiting member being for blocking said third proximal end portion of said third finger so as to maintain said third finger at the third open position.

32. The prosthetic hand as claimed in claim 8, further comprising a wrist frame, said main frame being rotatably mounted to said wrist frame, said drive unit further including a third transmission mechanism, said third transmission mechanism being coupled to said first transmission mechanism, said main frame and said wrist frame, said drive mechanism driving said first transmission mechanism and said third transmission mechanism to rotate said main frame relative to said wrist frame about a fourth axis that is orthogonal to said first axis between a first twist position and a second twist position.

33. The prosthetic hand as claimed in claim 32, wherein said wrist frame includes a central axle that defines the fourth axis, said main frame further including a support plate and an axial post, said support plate being rotatably mounted to said central axle, said axial post being mounted to said support plate, and being orthogonal to said central axle, said third transmission mechanism including a fixed gear, a movable gear, a first swing arm, a second swing arm and a link assembly, said fixed gear being fixedly mounted to said central axle, said movable gear being rotatably mounted to said axial post and meshing with said fixed gear, said first swing arm having an end co-rotatably mounted to said movable gear, said second swing arm having an end co-rotatably mounted to said first transmission shaft, said link assembly having two opposite ends that are respectively and pivotally connected to another end of said first swing arm and another end of said second swing arm, said first transmission shaft rotating said second swing arm to move said link assembly between a first move position and a second move position, so as to rotate said first swing arm and said movable gear, said main frame being at the first twist position when said link assembly is at the first move position, said main frame being at the second twist position when said link assembly is at the second move position.
